(19) United States
(12) Patent Application Publication
(10) Pub. No.: US 2025/0027868 A1
(43) Pub. Date: Jan. 23, 2025

(54) METHOD AND SYSTEM FOR ATOMIC INTERFEROMETRY

(71) Applicant: Technion Research & Development Foundation Limited, Haifa (IL)

(72) Inventor: Yoav SAGI, Haifa (IL)

(73) Assignee: Technion Research & Development Foundation Limited, Haifa (IL)

(21) Appl. No.: 18/775,072

(22) Filed: Jul. 17, 2024

Related U.S. Application Data
(60) Provisional application No. 63/527,090, filed on Jul. 17, 2023.

(51) Int. Cl. G01N 15/1434; G01V 7/02; G16C 10/00; G21K 1/00; H03L 7/26

(52) U.S. Cl. CPC G01N 15/1434; G16C 10/00; G21K 1/006; G01V 7/02; H03L 7/26

(57) ABSTRACT

An atomic interferometer system comprises optical tweezers each configured to trap at least one atom therein, an atom source system configured to release atoms, and a controller configured to control the optical tweezers to trap at least one atom released by the atom source system in one of the tweezers, to spatially split a wave function of the trapped atom between at least two of the tweezers, and to at least partially recombine the split atomic wave function in at least one of the tweezers. The atomic interferometer system can also comprise a measuring system configured to measure wavefunction population in each of the tweezers and to display an output pertaining to the wavefunction populations.

### RELATED APPLICATION(S)

This application claims the benefit of priority under 35 USC § 119 (e) of U.S. Provisional Patent Application No. 63/527,090 filed on Jul. 17, 2023, the contents of which are incorporated by reference as if fully set forth herein in their entirety.

### FIELD AND BACKGROUND OF THE INVENTION

The present invention, in some embodiments thereof, relates to a interferometry and, more particularly, but not exclusively, to a method and a system for atomic interferometry.

Atomic interferometers are specialized instruments that can accurately measure physical phenomena such as gravitational forces, acceleration, and rotation. They operate by creating a quantum superposition of two atomic wave packets that follow different paths and then interfere with each other. The resulting interference pattern reveals the relative phase shift accumulated during the motion, which is determined by the physical quantity being measured.

Known atomic interferometers employ splitting in momentum space. An example includes the Kasevich and Chu (KC) interferometer [17]. This interferometer uses stimulated Raman transitions to drive coherent Rabi oscillations between two momentum states, which then undergo different kinematic trajectories. Stopping the oscillation after a quarter of a period (π/2 pulse) generates a balanced superposition of these states. The KC interferometer is based on a sequence of π/2-π-π/2 pulses to achieve the splitting, mirroring, and recombining of the atoms, respectively. A knowing variant of the KC interferometer [40] combines trapping the atoms at the apex of a geodesic motion, reaching longer holding time.

### SUMMARY OF THE INVENTION

According to some embodiments of the invention the present invention there is provided an atomic interferometer system. The atomic interferometer system comprises: a plurality of optical tweezers each being configured to trap at least one atom therein; an atom source system configured to release atoms; a controller configured to control the optical tweezers to trap at least one atom released by the atom source system in one of the tweezers, to spatially split a wave function of the trapped atom between at least two of the tweezers, and to at least partially recombine the split atomic wave function in at least one of the tweezers; and a measuring system, configured to measure wavefunction population in each of the tweezers and to display an output pertaining to the wavefunction populations.

According to some embodiments of the invention each of the splitting and the recombination is adiabatic.

According to some embodiments of the invention the controller is configured to gradually increase a tunneling rate among at least two of the tweezers and to gradually decrease detuning energy among the at least two of the tweezers, to thereby effect the splitting.

According to some embodiments of the invention the controller is configured to gradually increase a tunneling rate among at least two of the tweezers and to gradually decrease detuning energy among the at least two of the tweezers, and then to gradually decrease the tunneling rate among at least two of the tweezers while maintaining a generally fixed detuning energy among the at least two of the tweezers, to thereby effect the splitting.

According to some embodiments of the invention the controller is configured to increase the tunneling rate and to decrease the detuning energy simultaneously.

According to some embodiments of the invention the controller is configured to effect the recombination a time period after the splitting, the time period being at least 10 seconds.

According to some embodiments of the invention the controller is configured control the tweezers in pairs in a manner that the splitting and the recombination are executed among each pair independently from other pairs.

According to some embodiments of the invention the atoms are fermionic atoms, wherein the atom source system is configured to release a plurality of atoms in a respective plurality of different discrete energy eigenstates, and wherein the controller is configured to control the optical tweezers to trap the plurality of atoms in one of the tweezers.

According to some embodiments of the invention the atom source system comprises a laser cooling system configured to cool the atoms to form a Fermi gas, and the controller is configured to trap the Fermi gas in in one of the tweezers and to reduce a number of atoms in the trapped Fermi gas by reduce a potential depth of the tweezer.

In some embodiments of the present invention the preparation scheme of the tweezer involves loading a relatively deep tweezer directly from a degenerate Fermi gas. To model the system, consider two boxes, large and small, which represent the degenerate Fermi gas and the tweezer, respectively (see FIG. 4). The number of states in the small box is such that, even when it is completely full, it has a negligible effect on the bog box. Assuming that the boxes are in thermal equilibrium with the same temperature T, and that particles move between them until their chemical potential, μ, is equal, the chemical potential in the large box is roughly given by μ≈E_{F}, where E_{F} is the Fermi energy of the large box. The Fermi energy in the small box is given by e_{F}≈μ+U=E_{F}+U, where U is the potential depth of the small box relative to the large box (the tweezer depth). The degeneracy in the small box is then given by T/t_{F}=T/(e_{F}/k_{B})=k_{B}T/(E_{F}+U), where (e_{F} is the Fermi energy in the small box. For example, when T=1 μK and T/T_{F}=2, the ratio T/t_{F} is about 0.01, assuming a tweezer with a depth of about 100 μK. At such low ratio T/t_{F}, the occupation probability of the low-lying eigenstates, given by n(E)=1/(exp(E−μ)/k_{B}T+1), is close to unity. After loading the tweezer, the highest eigenstates can be we eliminated by decreasing the tweezer's depth. The same reservoir can be used to load multiple tweezers, allowing for the operation of several interferometers in a single experimental run. This can be useful for improving the statistical signal to noise ratio and for studying transient phenomena.

In some embodiments of the present invention the atoms are alkali group atoms.

In some embodiments of the present invention the atoms are alkaline metal group atoms.

In some embodiments of the present invention at least one of the tweezers has a waist of less than 10 μm or less than 5 μm or less than 2 μm, e.g., about 1 μm in diameter.

According to an aspect of some embodiments of the present invention there is provided a system for measuring a Casimir-Polder force between an atom and a surface, comprising the atomic interferometer system as delineated above and optionally and preferably as further detailed below.

According to an aspect of some embodiments of the present invention there is provided a system for mapping subsurface structures, comprising the atomic interferometer system as delineated above and optionally and preferably as further detailed below.

In some embodiments of the present invention the atomic interferometer system is in use for measuring at least one of time, acceleration, rotation, and gravity.

According to an aspect of some embodiments of the present invention there is provided a system for detecting gravitational waves, comprising the atomic interferometer system as delineated above and optionally and preferably as further detailed below.

According to an aspect of some embodiments of the present invention there is provided a system for measuring atomic transitions, comprising the atomic interferometer system as delineated above and optionally and preferably as further detailed below.

According to an aspect of some embodiments of the present invention there is provided a system for seismic monitoring, comprising the atomic interferometer system as delineated above and optionally and preferably as further detailed below.

According to an aspect of some embodiments of the present invention there is provided an inertial navigation system, comprising the atomic interferometer system as delineated above and optionally and preferably as further detailed below.

According to an aspect of some embodiments of the present invention there is provided an atomic clock, comprising the atomic interferometer system as delineated above and optionally and preferably as further detailed below.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fec.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of an atomic interferometer system, according to some embodiments of the present invention.

FIG. 2 shows numerical simulation of a two-tweezer interferometer with a ⁴⁰K atom. On the left, the optical potential is plotted as faction of position (horizontal) and time (vertical). The tweezers have a waist of σ=1.3 μm and a final depth of 116 μK. The right tweezer starts with a detuning of Δ=−1.2 μK. The other four panels show the probability distribution |ψ(x)|² with different initial vibrational state n and different relative phases Δϕ between the arms. The black lines mark the tweezers paths. As shown, the splitting works regardless of the vibrational state, and the relative phase is translated into population difference in the output ports.

FIG. 3 shows two tweezers splitting and recombining process with π relative phase between the arms. (a) The potential parameters as a function of process time—the detuning between the tweezers normalized to the energy gap of the left tweezer at t=0 (blue line), and the distance between the tweezer centers normalized to the tweezer width (red line). (b) The spectrum for the process. The solid line represents the ground state energy, and the dashed line the first excited state energy. A line's or marker's color represents the wavefunction shape-green for an atom localized in a single tweezer (either left or right), blue for a symmetric 50:50 splitting and red for anti-symmetric 50:50 splitting. The markers depict the energy expectation value for a specific initial condition simulated. Left (right) pointing triangles are for atoms localized in the left (right) tweezer, and circles are for atoms split between the tweezers. At the beginning and the end of the process, both energy levels are localized at one tweezer. At the middle of the process (the splitting phase) the ground state becomes the symmetric 50:50 split, and the first excited level becomes the anti-symmetric 50:50 split. The dynamics depicted by the markers is of an atom initially localized at the left tweezer, adiabatically following the ground state until it is split between both tweezers with a symmetric wavefunction. At t=50 ms a π phase shift is applied between the interferometer arms, causing the wavefunction to become anti-symmetric. The atom proceeds to adiabatically follow the excited state until it is finally localized in the right tweezer.

FIG. 4 illustrates a process of loading tweezer directly from a degenerate Fermi gas at a temperature T and quantum degeneracy of k_{B}T/E_{F}. The phase space density is increased in the tweezer, yielding almost unit occupation probability in the low eigenstates in the case of only one spin.

FIG. 5 shows measurement of the Casimir-Polder force using tweezer atomic interferometer. Calculated relative phase shift due to the retarded CP potential with one arm at a 100 μm away from a metallic surface, and a second arm at a distance shown in the x-axis. The right y-axis shows the expected relative accuracy of this measurement. The inset shows two views of the sample and tweezer.

FIG. 6 is a schematic illustration of a method suitable for measuring the gravitational constant G. The tweezer cone angle is determined by the waist of the Gaussian beam, which in this case is σ=1.5 μm.

FIG. 7 is a schematic illustration of a scheme for interferometry with an atomic clock. The black solid lines represent the tweezers' centers in ID (vertical axis) as a function of time (horizontal axis). The upper trap is denoted as 1, and the lower trap as 2. Gray vertical lines represent the optical pulses driving the transitions between the clock states (internal degrees of freedom). The curly brackets indicate the time under the influence of a certain unitary transformation. The opacity and color of the shaded circles indicate the probability to find an atom at a particular spatial mode. Uₚₕₐₛₑ (T) marks the evolution for a duration T of the trapped atoms while they are subjected to the earth's gravitational potential. The vectors above the interferometer diagram are Bloch sphere vectors of the atomic states in the spin-½-like space spanned by the clock states at each corresponding time. The blue (red) arrows show the state at the upper (lower) arm.

FIG. 8 shows numerical simulation of the interferometric measurement with a separation of h=10 mm in Earth's gravitational field. The circular markers represent the average of the estimator of P_{g}, with error bars that represent the standard error. The solid line is a fit to P_{g}(ψ_{f}) as defined in EQ. (4) of Example 2.

FIGS. 9A and 9B show histograms from an ensemble of 1000 Monte Carlo simulation runs that include strong intensity fluctuations between the tweezer arms. In the incoherent splitting case (brighter shading), the wave packet was assumed to be randomly collapses to one of the arms after the splitting stage. For both Nₐ=10 atoms (FIG. 9A) and Nₐ=100 atoms (FIG. 9B) per run, the coherent and incoherent histograms are distinguished. The parameters in these simulations are T=10.00025 sec, Δ=2π·1000 Hz, h=10 mm, and N₂=5000. The different discretization of the x-axis is due to the different Nₐ, which results in a different finite set of possible values of P₁ in each run.

### DESCRIPTION OF SPECIFIC EMBODIMENTS OF THE INVENTION

The present invention, in some embodiments thereof, relates to a interferometry and, more particularly, but not exclusively, to a method and a system for atomic interferometry.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

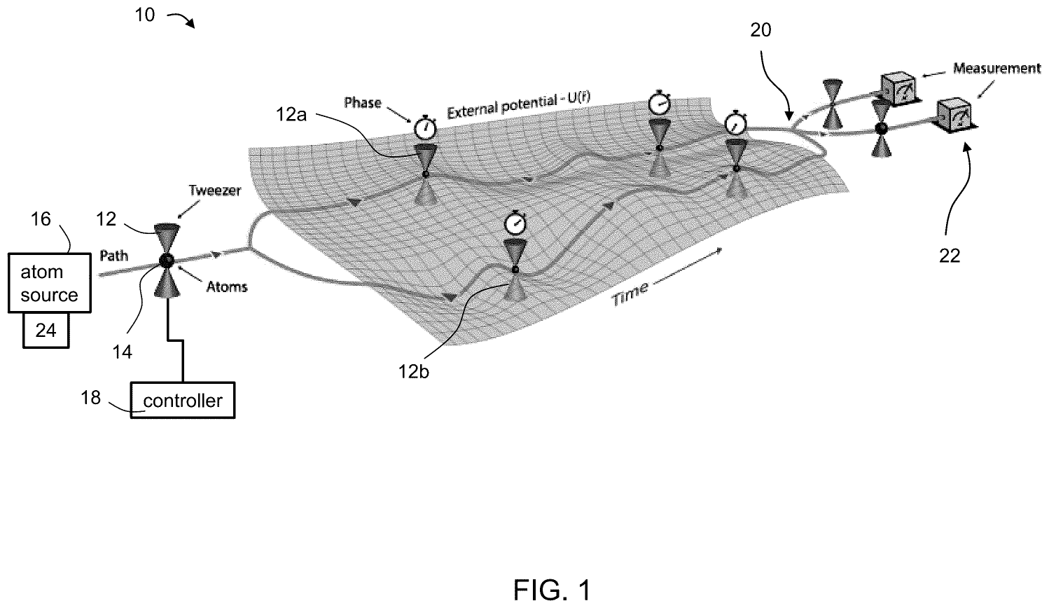

FIG. 1 is a schematic illustration of an atomic interferometer system 10, according to some embodiments of the present invention. System 10 comprises a plurality of optical tweezers 12 each configured to trap at least one atom 14 therein. While FIG. 1 illustrates an embodiment in which system 10 comprises two tweezers, 12a and 12b, the present embodiments contemplate use of any number of tweezers.

Each tweezer can be enacted by a focused light beam (e.g., a laser beam) that creates an electric field gradient in a manner that the intensity of the light is highest at a point that serves as the tweezer's center. Typically, the intensity of the light is highest at center of the beam. When the atom is placed in the laser beam, it experiences forces due to its interaction with the light. Due to the intensity gradient, the atom is trapped at the tweezer's center due to the force applied by the intensity gradient which pulls the atom towards the tweezer's center. In some embodiments of the present invention at least one of tweezers 12 has a waist of less than 10 μm or less than 9 μm or less than 8 μm or less than 7 μm or less than 6 μm or less than 5 μm or less than 4 μm or less than 3 μm or less than 2 μm, e.g., about 1 μm or less.

System 10 also comprises an atom source system 16 configured to release atoms. The atoms are optionally and preferably fermionic atoms. In this case, atom source system 16 optionally and preferably releases a plurality of atoms in a respective plurality of different discrete energy eigenstates. In some embodiments of the present invention atom source system 16 comprises a laser cooling system 24 configured to cool the atoms to form a Fermi gas. In some embodiments of the present invention an atomic number of the atoms released by atom source system 16 is at least 30. For example, the atoms can be alkali group atoms (e.g., ⁴⁰K). Alternatively or additionally, the atoms can be and in lanthanide group atoms (e.g., ¹⁷¹Yb). Alternatively or additionally, the atoms can be alkaline metal group atoms.

System 10 also comprises a controller 18 configured to control optical tweezers 12 to trap one or more atoms released by atom source 16 system in one of tweezers (e.g., tweezer 12a). When atom source system 16 releases a plurality of atoms in a respective plurality of different discrete energy eigenstates, controller 18 controls optical tweezers 12 to trap the plurality of atoms in one of the tweezers (e.g., tweezer 12a). When source 16 releases a Fermi gas, controller 18 optionally and preferably traps the Fermi gas in one of the tweezers (e.g., tweezer 12a) and reduces the potential depth of the tweezer, so as to allow the atoms occupying the highest eigenstates to escape from the respective tweezer thus controlling the number of atoms in the tweezer.

Controller 18 is also configured to spatially split a wave function of the trapped atom or atoms between two or more of the tweezers (e.g., between tweezers 12a and 12b), and to at least partially recombine 20 the split atomic wave function in at least one of the tweezers. System 10 further comprises a measuring system 22 that measures the wavefunction population in each of the tweezers and displays an output pertaining to the measured wavefunction populations. Measuring system 22 can be of any known type that allows measuring a population of a wavefunction in an optical tweezers. For example, measuring system 22 can be configured to apply a technique selected from the group consisting of fluorescence or absorption imaging, e.g., by means of an EMCCD camera, fluorescence counting, state-selective detection, and the like.

In some embodiments of the present invention the splitting and/or the recombination, and more preferable both the splitting and the recombination, is adiabatic.

As used herein, “adiabatic splitting” means a splitting process during which the eigenstates evolve continuously but the atomic wave packet projection on the eigenstates does not change during the process.

As used herein, “adiabatic recombination” means a recombination process during which the eigenstates evolve continuously but the atomic wave packet projection on the eigenstates does not change during the process.

In some embodiments of the present invention controller 18 spatially splits wave functions by first gradually increasing the tunneling rate among two or more of tweezers 12 and gradually decreasing a detuning energy among tweezers 12. Preferably, following the gradually decrease in the detuning energy, controller 18 gradually decreases the tunneling rate among two or more of tweezers 12, and optionally and preferably gradually increasing the detuning energy among tweezers 12, thus effect the wavefunction split. The time-dependence of the tunneling rate is denoted J(t), and it can be controlled by varying the spatial distance between the tweezers. For example, when the spatial distance between the tweezers is sufficiently large to ensure that there is no tunneling between them, J(t)=0, and when the spatial distance between the tweezers is sufficiently small to ensure that there is tunneling between them, J(t)>0.

The time-dependence of the detuning energy is denoted herein by hΔ(t), where h is the reduced Planck constant, and Δ(t) is a time-dependent detuning parameter that is varied with the time t by the controller. In some embodiments of the present invention Δ(t) varies linearly with the time during at least a portion of the splitting process, more preferably during the entire splitting process. The value of Δ(t) can be controlled by adjusting the relative intensities between the light beams that enact the tweezers. Specifically, when the light beams have the same intensity, hΔ(t)=0 and when the light beams have different intensities, hΔ(t)≠0. The parameter Δ(t) can be selected such that when it is positive the potential of the initially empty tweezer is shallower, and when it is negative the potential of the initially occupied tweezer is shallower.

In an example embodiment, the splitting process begins with a configuration in which only one of the tweezers holds the atom, while other tweezers are empty, wherein the spatial distance between the occupied and empty tweezers is sufficiently large to ensure that there is no tunneling between them, thus ensuring that J(t)=0 at the initial stage. At this stage, hΔ(t) can be set to a non-zero value (e.g., positive) by ensuring that the light beams have different intensities. The splitting process continues by decreasing the distance between the empty and occupied tweezers, thus gradually increasing J(t), so that there is tunneling from the occupied tweezer to the empty tweezer. At this stage, the absolute value of the detuning parameter is lowered to zero. Thereafter, the distance between the tweezers is increased again, while maintaining Δ(t)=0, thus effecting a wavefunction split among the tweezers. Preferably, controller 18 increases the tunneling rate and decreases the detuning energy simultaneously, so that the distance between the tweezers is decreased while adjusting the intensities of the light beam to ensure that the absolute value of the detuning parameter is decreased.

In some embodiments of the present invention controller 18 recombines the wave functions by time-reversing the splitting process. The recombination is preferably effected at least 10 seconds or at least 15 seconds or at least 20 seconds or at least 25 seconds or at least 30 seconds or at least 35 seconds or at least 40 seconds after the splitting.

In some embodiments of the present invention controller 18 is configured control the tweezers in pairs in a manner that the splitting and recombination are executed among each pair, independently from other pairs.

System 10 can be used in any one of a variety of applications, including, without limitation, measuring a Casimir-Polder force between an atom and a surface, mapping of subsurface structures, measuring time, acceleration, rotation, and/or gravity, detecting gravitational waves, measuring atomic transitions, seismic monitoring, inertial navigation. In some embodiments of the present invention system 10 serves as an atomic clock.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

The Inventor found that atomic interferometers in which the wave-packet splitting is in momentum space suffer from severe constraints on the possible atomic trajectory, positioning accuracy, and probing duration. This Example described atomic interferometer that uses micro-optical traps (optical tweezers) to manipulate and control the motion of atoms. The interferometer of the present embodiments allows long probing time, sub-micrometer positioning accuracy, and flexibility in the shaping of the atomic trajectory. The interferometer of the present embodiments provides coherent atomic splitting and combining schemes. This example presents adiabatic schemes that are robust in the presence of experimental imperfections and work simultaneously with many vibrational states. The latter property allows for multiatom interferometry in a single run. This Example explains the advantage of using fermionic atoms to obtain single-atom occupation of vibrational states and to eliminate mean-field shifts. The sub-micrometer resolution and extended measurement duration allows exploring fundamental physical laws in new regimes.

### Introduction

Interferometers have a long history of driving scientific revolutions, from the Michelson-Morley experiment, to the recent observation of gravitational waves [1]. Soon after the discovery of the wave-particle duality, in the early years of the 20th century, it was realized that the interference of massive particles could be harnessed for the purpose of highly precise measurements. Over the years, matter-wave interference has been demonstrated using a wide range of masses, including electrons, atoms, and complex molecules [10,15]. The development of laser cooling techniques has made cold atoms a popular choice for interferometry due to their large de Broglie wavelengths and slow velocities, which allow for long coherence and integration times.

Atomic interferometers (AIFs) come in many forms, but they all rely on the same fundamental principle; the atomic wave packet is initially prepared in a specific state and then coherently divided into two parts that follow distinct paths. The quantum wave function in each arm may acquire a different phase. The two arms are then coherently combined, and the process of recombination maps the relative phase shift between them to populations in two output states, which may be external (e.g., spatial modes, momentum states) or internal (e.g., spin projections, atomic energy levels). The most significant distinction between atomic and photonic interferometers is the non-zero mass of the former. This means that in an atomic interferometer, atoms can be brought to a complete halt.

To coherently split wave functions of atoms, AIFs have initially employed diffraction from periodic fabricated structures [21,22] and optical lattices [25]. These techniques utilize the exchange of lattice momentum to create sidebands in the atomic wave function's momentum distribution, which leads to a coherent splitting of the atomic path in real space. They are relatively simple to implement and robust, but the Inventor found that they have low efficiency and limited control over the atomic trajectory. A different approach is to utilize the coherent absorption of a single photon [5] or two photons with different wave vectors [8] to generate coherent splitting in momentum space. One well-known example is the Kasevich and Chu (KC) interferometer [17], which uses stimulated Raman transitions to drive coherent Rabi oscillations between two momentum states, which then undergo different kinematic trajectories. Stopping the oscillation after a quarter of a period (π/2 pulse) generates a balanced superposition of these states. The KC interferometer is based on a sequence of π/2-π-π/2 pulses to achieve the splitting, mirroring, and recombining of the atoms, respectively.

KC interferometers were instrumental in many precision measurements over the past two decades, including determination of the gravitational constant [12, 30], measurement of the fine-structure constant [29], testing the equivalence principle [3], and constraining dark energy models [14]. However, the Inventor found that they suffer from several shortcomings, including limited spatial resolution and atomic motion which is geodesic only (e.g., free fall) and cannot be freely shaped. In particular, the Inventor found that it is not possible to position the atoms at rest at arbitrary locations. Moreover, to have a long probing duration, the experimental apparatus tends to be very large, and even then, the interaction time is limited to a few seconds.

Recently, the group of Müller developed a variant of a KC interferometer that combines trapping the atoms at the apex of the geodesic motion, reaching a holding time of 20-60 s [40]. However, the atomic motion was still ballistic, and the maximum separation between the wave packets was tens of micrometers. Matter-wave interference with 3D-confined condensates of bosons and formionic pairs was also demonstrated, but only as a tool to study the coherence of the condensate wave function [32,37]. Specifically, substantial stochastic phase shifts due to inter-particle interactions in these gases make them unsuitable for precision metrology. Interferometry with a single trapped atom was demonstrated in a spin-dependent lattice [35]. However, the maximum separation was around 10 μm, and the holding time was limited to around 1 ms due to spontaneous scattering from the lattice.

This Example describes atomic interferometry that employs mobile micro-optical traps, known as “optical tweezers,” to trap and manipulate individual atoms. Progresses in this field are described in [4, 6, 13, 19, 27, 33, 41]. Neutral atoms in optical tweezers have been used in quantum computing as qubits, in quantum simulation of many-body phenomena, and for precision time measurements. According to the present embodiments the tweezers are used for coherently splitting and recombining the atoms and, in between, holding the atomic wave packets for tens of seconds with sub-micrometer positioning accuracy and complete freedom to shape the atomic trajectory. The advantage is the implementation of atomic splitters and combiners that do not change the internal state of the atom, are robust in the presence of experimental imperfections, and work with many vibrational states of the tweezer. Fermionic atoms are used and their Fermi-Dirac statistics are leveraged to have between a few tens of atoms and a hundred atoms in a single run while avoiding systematic interaction energy shifts. This unique combination allows high-precision measurement of potentials with sub-micrometer resolution.

### Tweezer-Based Atomic Interferometer

A schematic sketch of the interferometer of the present Example is shown in FIG. 1. An atomic wave-packet is prepared and held in one of the optical tweezers. Then, the wave-packet is split coherently into the two tweezers, each moving in a different path, and afterwards it is recombined. By interfering the two wave-packets, the relative phase shift between the arms, which arises due to differences in the external potential and dynamics along the paths, is detected. Typically, experiments with ultracold atoms are conducted in an ultra-high vacuum chamber, allowing to hold the atoms for a very long duration (i.e., tens to hundreds of seconds). The optical tweezers can have a waist, σ, of about 1 μm, and the atomic wave-function is typically localized to around 100 nm. Combining this with a positioning precision of few hundreds of nanometers yields a spatial resolution better than a micrometer. In some embodiments, the atomic splitter-combiner is adiabatic, and in some embodiments the atoms are fermions. The fermionic statistics guarantees that each energy eigenstate of the tweezer (“vibrational” states) is occupied by only a single atom and that the atoms do not interact.

As used herein, “adiabatic recombination” means a recombination process during which the eigenstates evolve continuously but the atomic wave packet projection on the eigenstates does not change during the process.

The tweezer can be loaded from a moderately degenerate Fermi gas (e.g., T/T_{F}≈1, where T_{F} is the Fermi temperature), harnessing the “dimple effect” to enhance the phase space density [31, 34]. Thus, the occupation probability in all low-lying eigenstates can be very close to unity. After loading the tweezer, the atoms occupying the highest eigenstates are eliminated by gradually reducing the tweezer's depth, until the desired number of atoms is reached. The Pauli exclusion principle ensures that at each vibrational state there is at most one atom at a specific spin state. By loading the tweezer from a spin-polarized Fermi gas, or alternatively by employing a magnetic field gradient, only a single atom is prepared per vibrational state. Preferably, the atoms are with a relatively high mass. This is advantageous since in this case the interferometer is more sensitive to acceleration and gravitational potential. In the alkali group, ⁴⁰K is preferred, and in the lanthanide group, ¹⁷¹Yb is preferred [16, 23].

Preferably, the tweezer is operated with as many atoms as possible so as to reduce the number of repetitions needed to achieve a certain level of uncertainty. Additionally, having many atoms in a single run allows one to measure transient phenomena which cannot be averaged. Multi-atom tweezer interferometry can be achieved in more than one way. In some embodiments, several independent tweezers are employed in parallel, each operated as a single-atom interferometer. The number of independent tweezers that can be employed can be from about 10 to about 1000, or from about 20 to about 1000, or from about 40 to about 1000, or from about 80 to about 1000, or from about 40 to about 500, or from about 40 to about 500, or from about 40 to about 250 tweezers at the same time. The tweezers are preferably micrometer-sized, allowing fitting many of them in a small area and still keep the distance between them large enough to avoid interaction shifts. The detection is optionally and preferably performed in parallel by optical imaging with a sensitive camera.

Multi-atom tweezer interferometry can be achieved alternatively or additionally by utilizing the many bound vibrational states of a single tweezer. As demonstrated below, splitting and recombining schemes work successfully for many vibrational states. This property allows for multiple atoms to be used in the interferometer at the same time [2]. The Inventor found that the combiner maps differential phase shifts between the interferometer arms to population differences in the output ports in a manner that does not depend on the vibrational state. This is advantageous because it allows executing a single run of the method using N atoms in a single tweezer instead of using N tweezers with a single atom each. In some embodiments the number of atoms with vibrational states that can be used in a single tweezer is from about 10 to about 1000, or from about 20 to about 1000, or from about 40 to about 1000, or from about 80 to about 1000, or from about 40 to about 500, or from about 40 to about 500, or from about 40 to about 250. This significantly increases the signal-to-noise ratio compared to a single-atom interferometer. For example, 100 atoms populating the same tweezer with different vibrational states can yield a 10-fold improvement in the signal-to-noise ratio. The Inventor found that the splitting does not change the atoms' internal state, making the superposition robust to spin-dependent noise. Since the atoms are fermionic, interaction between the atoms is precluded by formionic anti-symmetry, thus avoiding systematic shifts.

### Coherent Splitting and Recombining

The interferometer of the present embodiments splits and merges the atomic wave-packets coherently. In the present example, two tweezers are employed. A tweezer interferometer based on the two-tweezer scheme is somewhat similar the optical Mach-Zehnder interferometer.

The process preferably involves at least two input and output ports, and is preferably reversible, meaning that two consecutive executions of the process bring the atoms back to the original port. In some embodiments, the process is designed such as to favor detection of population in two spatially separated tweezers over distinguishing between the population in two different vibrational states of the same tweezer. The process of the present embodiments preferably withstands small variations in parameters, such as duration, tweezer intensity, and position. In embodiments in which the same tweezer is populated with multiple atoms occupying different vibrational states, the process is selected to be insensitive to the vibrational state.

Consider, for example, a simple π/2 splitter. The initial state is one tweezer occupied by a single atom in some vibrational eigenstate and a second empty tweezer at a close proximity such that there is tunneling. As time progresses, the atom undergoes oscillations back and forth between the two tweezers [18]. If the dynamics is ceased exactly in the middle of such an oscillation (e.g., by moving the tweezers apart), the atomic wave packet coherently divides between the two tweezers. While this scheme, which is analogues to a π/2 pulse in Rabi oscillations, can be used as a splitter, the Inventor found that it is advantageous to improve it because the splitting is first-order sensitive to changes in the process duration, because the tunneling rate is strongly dependent on the distance, so that positioning noise may result in fluctuation in the splitting process, and because due to the strong distance dependence the scheme is suitable mainly for the case of a single state.

Following is a description of an adiabatic driving scheme that is more robust to experimental imperfections and vibrational state occupation.

### Exemplified Two-Tweezers Atomic Splitter

In this Example, the splitting scheme is an implementation of a rapid adiabatic passage [38]. It starts with two tweezers, one holding the atoms and the other is empty. Initially, the two tweezers are positioned at a large enough distance such that there is no tunneling between them. There are two parameters which are varied in time: the energy detuning between the tweezers, hΔ, and the tunneling rate, J. The former can be controlled by adjusting the relative power between the beams, and the latter can be controlled by varying the distance d(t) between the tweezers. At t=0, J=0 and Δ is set to a small positive value, which means that the potential of the empty tweezer is shallower. The protocol is performed by increasing J(t) (e.g., by moving the tweezers such as to decrease the distance d(t)), while lowering the detuning parameter Δ(t). Preferably, the tunneling rate is increased simultaneously with the lowering of the detuning parameter. At time tₘ, when the tweezers are closest to each other, Δ(t) is preferably at its minimal value, e.g., zero. Then, the distance (t) is increased again, while maintaining Δ(t) at its minimal value (e.g., maintaining Δ(t)=0.

The protocol splits the atomic wave function evenly between the two tweezers. To show this, a tight binding model is employed. For simplicity, only one vibrational eigenstate is considered in each tweezer. One of ordinary skills in the art, provided with the details described herein would know how to adjust the model to the case of vibrational eigenstates per tweezer. The vibrational eigenstate is denoted |φ [symbol] , and its energy is denoted Eᵢ, where i={1,2} identifies the tweezer. The Hamiltonian of this system, in the rotating wave approximation, can be written as

$$H = ℏΔ \vert {φ}_{2} 〉 〈 {φ}_{1} \vert + ℏ \frac{J}{2} \vert {φ}_{2} 〉 〈 {φ}_{1} \vert + h . c .. \qquad ( EQ . 1 )$$

The state of the system can be described using a Bloch vector \vec{ν}=( [symbol] σₓ [symbol] , [symbol] σ_{y} [symbol] , [symbol] σ_{z} [symbol] ), where σᵢ are the Pauli matrices operating in the two-dimensional subspace of {φ₁, φ₂}. The dynamics of the system is given by the optical Bloch equation:

$${v}^{\dot{\to }} = \times \vec{v} ,$$

where [symbol] is the torque vector around which the Bloch vector performs precession. The initial state is |ψ [symbol] =|φ₁ [symbol] . The initial detuning is chosen Δ≈ω, where ω is the tweezer oscillation frequency. This choice is made to have the largest possible initial Δ before eigenstates with different vibrational numbers cross. These initial conditions correspond to the Bloch and torque vectors being parallel, each pointing towards one of the poles. When the tweezers are gradually brought closer and the detuning is changed to Δ→0, the torque vector rotates to the equatorial plane and the Bloch vector follows adiabatically. The scheme ends with a gradual decrease of J→0, leaving the Bloch vector in the equatorial plane. This means that the wave function is

$$\vert ψ 〉 = \frac{1}{\sqrt{2}} [ \vert {φ}_{1} 〉 + \vert {φ}_{2} 〉 ] ,$$

as desired. Note that because the process is adiabatic it works with any initial eigenstate that fulfills the adiabatic condition.

To test the performance of this splitting scheme beyond the two-level approximation, the Inventor employed numerical solutions of the time-dependent Schrödinger equation using the split-step Fourier method [39]. Because the coupling is predominantly in the radial direction, the system was modeled in one dimension. Nonetheless, the findings were confirmed in a two-dimensional setting.

In this Example, the simulation were employed using a trap potential V (x, t) that included two Gaussian tweezer beams:

$$V ( x , t ) = - {V}_{0} [ {e}^{- 2 \frac{x - d ( t ) / 2}{{σ}^{2}}} + ( 1 - Δ ( t ) ) {e}^{- 2 \frac{x + d ( t ) / 2}{{σ}^{2}}} ] . \qquad ( EQ . 2 )$$

The time-dependences of the distance, d(t), between the tweezers, and of the detuning parameter Δ(t), were selected to be:

$$d ( t ) = \frac{1}{2} ( {d}_{max} + {d}_{min} ) + \frac{1}{2} ( {d}_{max} - {d}_{min} ) cos ( 2 π t / T ) \\ Δ ( t ) = \{ \begin{matrix}{Δ}_{max} ( 1 - 2 t / T ) & if t < T / 2 \\ 0 & if t \geq T / 2\end{matrix} ,$$

where T is the total splitting process time, dₘₐₓ and dₘᵢₙ are the initial and shortest distances between the centers of the tweezers, respectively.

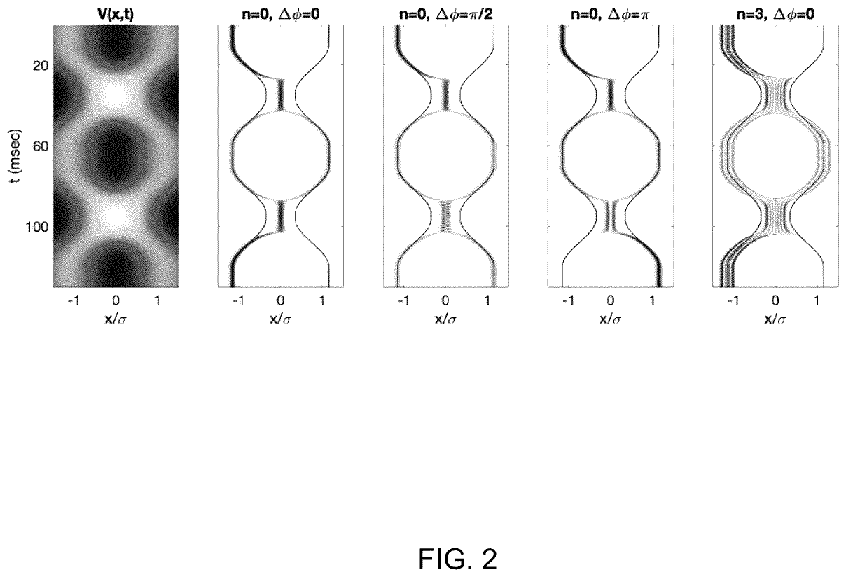

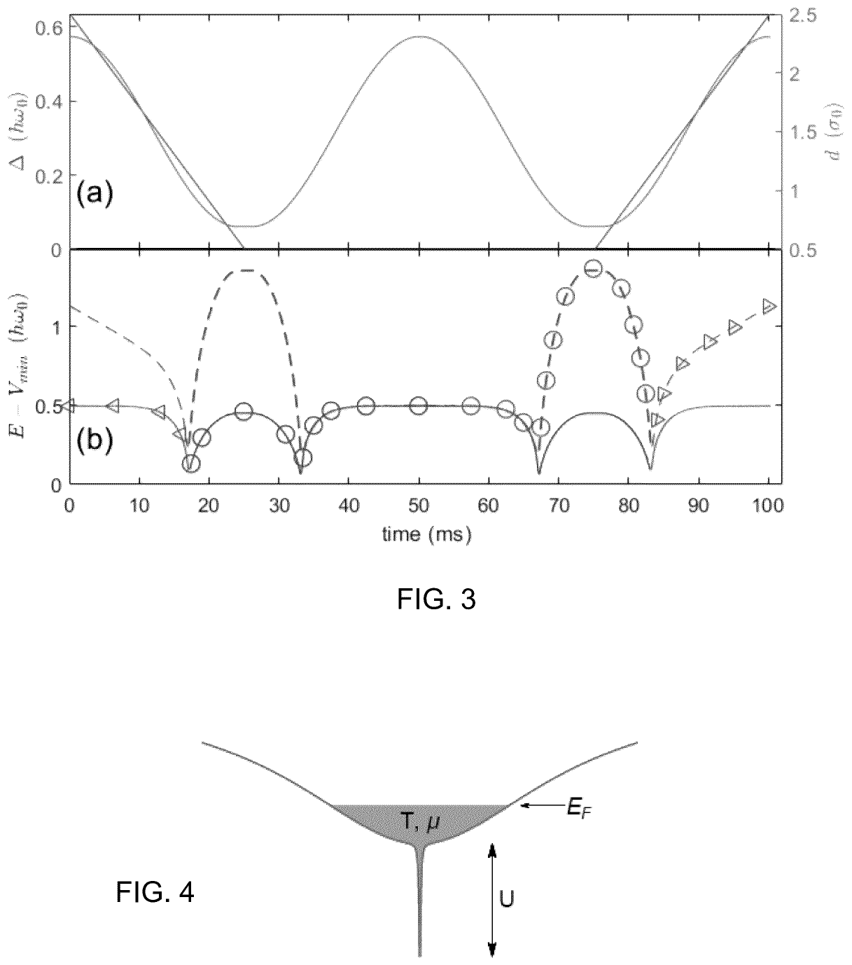

The results are shown in FIG. 2. The initial wave-function was a specific eigenstate of one of the tweezers, calculated by numerical diagonalization of the Hamiltonian of that specific potential. There are two observables that were calculated at the end of the simulated process: the probability to be in a given tweezer, and the overlap fidelity of the wave function with the initial eigenstate. The overlap fidelity is defined as:

$${f}_{right ( left )} = {\int }_{- ∞}^{∞} {ψ}^{*} ( x ) {φ}_{2 ( 1 )} ( x ) dx .$$

The time steps and spatial resolution in the simulation were selected chosen to ensure convergence of these observables. The process parameters were optimize to get as close as possible to unit fidelity and a probability of ½ in each tweezer. The simulations demonstrate that the relative phase between the arms is correlated with the population difference between the output tweezers. It also shows (second and last panel of FIG. 2) that the interferometric loop works the same for both ground and excited vibrational states.

The numerical simulations reveal surprising aspect of the splitting scheme. The surprising aspect is that it works successfully even when the minimal distance between the tweezers is small enough that there is only a single minimum to the combined potential. In this regime, where the tweezer potential is merged, the tight-binding approximation does not hold, and since there is no barrier, the notion of tunneling needs to be reconsidered. The reason why the splitting scheme still works is because it is based on adiabatic following which can be generalized to merged potentials. Initially, when the tweezers are far and the occupied tweezer has a lower energy, the occupied state is essentially identical to the eigenstate of only a single tweezer. Then, when the tweezers are brought closer and the detuning is reduced to zero, this state evolves adiabatically to the symmetric state of the two tweezers. This adiabatic passage is protected by an avoided level crossing that opens a gap, which evolves from 2 ℏJ, when the tweezers are only weakly coupled, to ℏω_{osc}, when they are overlap (ω_{osc} is the harmonic frequency of the combined potential). Note that if the initial Δ is set to a negative value instead of a positive value, then the adiabatic following is of the excited state, and the final state of the atom is the anti-symmetric wave function. In this case, there is a π phase between the arms of the interferometer.

Once the splitting process is completed, the two output tweezers are taken apart. At this stage, the gap is closed, and the symmetric and anti-symmetric states of the two tweezers become essentially degenerate. This fact is advantageous, since the phase shift between the atomic wave packets is translated into a specific mixing between the symmetric and anti-symmetric states. Then, the time-reversed version of the splitting process achieves a coherent combining, where the differential phase shift becomes the relative population between the two tweezers exiting the combiner (see FIG. 2). To understand this process, note that each of the symmetric and anti-symmetric states evolves adiabatically and eventually becomes a state localized in the left or right tweezer.

### Sensitivity and Precision Estimation

The evolution of the wave function is given by |ω(t) [symbol] =e^{-iS}^{_{Γ}}^{/h}|ω(t=0) [symbol] , where S_{Γ} is the action, defined by the integration over the Lagrangian along the classical path Γ:

$${S}_{Γ} = {\int }_{Γ} ℒ [ r , \dot{r} ] dt$$

(see [10, 36]). In the interferometer of this Example, defined by two paths Γ₁ and Γ₂ for the two arms, the relative phase shift acquired by an atom is therefore:

$$Δϕ = \frac{1}{ℏ} {\int }_{{Γ}_{2}} ℒ [ r , \dot{r} ] dt - \frac{1}{ℏ} {\int }_{{Γ}_{1}} ℒ [ r , \dot{r} ] dt .$$

To assess the sensitivity of the tweezer interferometer, a simple scenario in which the atom is split symmetrically and separated to a distance h, where it is held at rest, and then recombined, is considered. The movements are assumed to be symmetric and short compared to the total measurement duration, R, and therefore their contribution to the phase difference are not included.

Consider for simplicity that the interferometer is subjected to a uniform acceleration a (e.g., gravity) aligned parallel to the line connecting the two tweezers. Then, the action can be written as

$$ℒ = - m \cdot x \cdot a ,$$

where m and x are the atom's mass and position along this line. The phase difference is Δϕ=(m·h·a/ℏ)T. The lifetime of atoms in optical tweezer can be many tens of seconds. Using acousto-optics deflector (AOD) technology, the distance between tweezers can be tuned up to hundreds of micrometers. The distance can be further increased if the tweezers are generated by two separate AODs, steered by two piezo-controlled mirrors, and then combined with a beam splitter. This optical scheme allows for precise control at short distances using the AOD and reaching large distances with the piezo mirrors. Thus, the distance between tweezers is only limited by the objective field of view. The Inventors estimated that a separation of 10-50 mm can be reached. Taking T=10 s and h=10 mm, a phase shift of Δϕ≈6.2·10⁸ rad is obtained for earth gravitational acceleration a=g. By increasing the distance between the tweezers to 50 mm and the waiting time to a minute, the phase difference is increased to about 2·10¹⁰ rad.

In comparison, in a conventional Kasevich-Chu atomic interferometer the phase shift is given by Δϕ=k_{eff} g T², with k_{eff} being the effective wave-vector of the momentum kick given to split between the two arms [11]. Taking as typical numbers k_{eff}=4π/(780 nm) and T=1 s [30], one obtains Δϕ≈1.6·10⁸ rad for the gravitational accelerations. More advance versions of the Kasevich-Chu interferometer can impart a larger momentum kick of few tens to a hundred ℏk (k in the Raman laser wave vector), but this comes with a price of large sensitivity to wave front distortions and phase noise of the Raman beams [9].

It is preferred that the two tweezers have the same or similar intensity (e.g., with deviation of less than 30% or less than 20% or less than 10% or less than 5%). Thus, as long as intensity fluctuations are approximately common to both tweezers, they do not introduce relative phase noise. Large relative fluctuations, on the other hand, lead to a relative phase shift, thereby impairing the interferometer's operation. A fundamental source of such relative intensity fluctuations will now be considered. This is the shot noise of each beam. Denote the peak power of each tweezer as P₀. The average number of photons in each tweezer during the experiment is N=TP₀/ℏωₜ, where ωₜ is the tweezer laser angular frequency. The relative phase noise is given by δφ/Φ=√2·δN/N, which for a shot noise is √2(N)^{-1/2}. Note that here Φ is the phase acquired in each tweezer due to the optical potential and not due to the external potential. Thus, one obtains

$${( \frac{δϕ}{Φ} )}_{shot noise} = \sqrt{\frac{2 {ℏω}_{t}}{{TP}_{0}}} .$$

As an example, a ⁴⁰K atom held for 10 s in a tweezer interferometer, where each arm has a power of 100 μW and a Gaussian waist of σ=1 μm is considered. This yields a tweezer depth of approximately 8.6 μK, for which δϕ/Φ≈2·10⁻⁸ and δϕ≈0.2 rad. It is difficult to mitigate all the technical noise sources such that the limiting factor is the shot noise.

Next, the sensitivity to noise of the splitting and recombining stages is examined. It is assumed that the differential noise between the tweezers has reached the shot noise limit. The Inventors found numerically that at this level, this noise has no measurable effect on the performance of the splitter or combiner. However, common mode fluctuations of the tweezer intensity may still exist. To assess their impact, the Inventors run simulations where random variations with zero mean, Vₙ(t) were introduced to the tweezer amplitudes. Vₙ(t) is characterized by white noise with spectral density S(f)=η2ℏωₜP₀, which is η times larger than the shot noise. It is assumed that the common-mode noise amplitude can surpass the shot noise amplitude significantly, by approximately 70 decibels (dB). It is additionally assumed that this noise is uniformly applied to the tweezers.

For every noise realization, a complete simulation of the interferometer process was performed, and the probabilities of locating the atom in each of the output arms were determined.

Four different types of interferometry experiments were considered. The experiments varied in total duration, number of atoms per run, and number of repetitions per phase as summarized in Table 1, below.

TABLE 1
Different types of experimental scenarios with a tweezer-based interferometer

| Scenario number | No. of repetitions | No. of atoms in each run | T [sec] | total runtime |
|---|---|---|---|---|
| 1 | 10 | 1 | 10 | ~50 min |
| 2 | 10 | 10 | 10 | ~50 min |
| 3 | 285 | 100 | 10 | ~24 hr |
| 4 | 66 | 100 | 60 | ~24 hr |

In each scenario, 20 equally spaced phases were selected to simulate a “fringe scan” and the phase shift between the arms, which is due to the physical phenomenon under investigation was determined. For each of the 20 phases, a random noise of 0.2 rad was added to account for shot noise (0.49 rad for T=60 sec). This is justified if during the measurement time T the tweezers' amplitudes were lowered to 8.6 μK and were then increased them back before the combiner stage. If multiple atoms are involved in a run, they all experience the same random noise, indicating that they are exposed to the same noise realization. The closest phase out of the 4000 realizations was select for each run and its calculated quantum wave function was used to determine the output probabilities. These probabilities were used to randomly assign an exit port for each atom. The numerical data were fitted and the phase in the presence of noise and its error relative to the known physical phase were determined. This procedure allowed to determine the expected accuracy of the interferometer in various realistic scenarios.

The results are presented in Table 2, below.

TABLE 2
Simulation results-error estimation for atomic interferometer

| | phase accuracy [mrad] | | | |
|---|---|---|---|---|
| η | scenario 1 | scenario 2 | scenario 3 | scenario 4 |
| 1 | 87 | 34 | 4 | 18 |
| 10⁷ | 93 | 34 | 4 | 18 |
| 5 · 10⁷ | 195 | 105 | 37 | 49 |

As demonstrated, the phase accuracy of the device remains largely unaffected in a wide range of noise levels. The primary source of error is the statistical quantum error, unless the experimental scenario involves numerous repetitions/atoms. In such cases, the accuracy is determined by the minor phase error induced by shot noise over time T.

The accuracy of the interferometer deteriorates at a particular noise level due to an uneven division of the waveform caused by the splitter. Yet, the noise levels at which this decrease in accuracy occurs are exceptionally high and significantly exceed those typically encountered in commercial lasers, where n ranges from 10³ to 10⁴.

### Discussion

The tweezer-based atomic interferometer of the present embodiments can be used in many fields where a sensitive force probe with sub micrometer resolution is needed. This Example discusses two such applications: measurements of the gravitational constant and surface forces. Another example is mapping gravitational forces at short distances for testing non-Newtonian gravity theories [20, 26, 42]. The interferometer of the present embodiments can also be utilized to search for quantum gravity effects, e.g., detection of entanglement between two different configurations of an atom and a mechanical oscillator [7]. Another application which can benefit from the interferometer of the present embodiments is the study of material properties in condensed matter. Specifically, the tweezer interferometer can be used to measure forces of localized topological defects such as vortices or skyrmions. It can also map with unprecedented sensitivity magnetic fields near surfaces.

### Example 2

Clock interferometry refers to the coherent splitting of a clock into two different paths and recombining in a way that reveals the proper time difference between them. Unlike the comparison of two separate clocks, this approach allows testing how non-flat spacetime influences quantum coherence. Atomic clocks are currently the most accurate time keeping devices. This Example describes the use of the optical tweezers of the present embodiments to implement clock interferometry. The clock interferometer of this Example employs an alkaline-earth-like atom held in an optical trap at the magic wavelength.

The term “magic wavelength” refers to a specific wavelength for which both clock states experience the same trap potential, e.g., the potential V(x,t) given by EQ. (2) of Example 1.

Through a combination of adiabatic, tweezer-based, splitting and recombining schemes and a modified Ramsey sequence on the clock states, a linear sensitivity to the gravitational time dilation is achieved. The measurement of the time dilation is insensitive to relative fluctuations in the intensity of the tweezer beams. The interferometer described herein can test the effect of gravitational redshift on quantum coherence, and implement the quantum twin paradox.

This Example describes an atomic clock interferometry (ACIF) scheme that uses optical pulses only to create a balanced superposition of the clock states in each arm, while the splitting process is achieved by using adiabatic tunneling transitions between optical tweezers. The scheme achieves linear sensitivity to gravitational time dilation, providing a significant signal in realistic experimental timescale. Importantly, we show that when the tweezer is at a magic wavelength, the interference signal is insensitive to relative intensity fluctuations between the tweezers. This property makes an interferometric measurement of the gravitational red shift with our proposed scheme feasible with current technological capabilities. The significance of this experiment lies in measuring a general gravity effect with a spatially separated coherent quantum state for the first time.

### Tweezer Clock Interferometry

According to general relativity, the proper time, τ, is determined by the metric, g_{μν}, according to

$$τ = {\int }_{Γ} d τ = \frac{1}{{c}^{2}} {\int }_{Γ} \sqrt{{g}_{uv} {dx}^{μ} {dx}^{v}} ,$$

where the integration is performed along a path Γ. Clocks that follow different paths may experience a difference in their proper time. In particular, if clocks are positioned at different locations under the influence of a gravitational field, they tick at a different rate. This prediction of general relativity was confirmed using two clocks at different heights. On the other hand, proper time is connected to matter-wave phase through:

$$ϕ = \frac{{mc}^{2}}{ℏ} τ ,$$

where m is the mass, and c is the speed of light. This serves as a motivation for using a matter-wave interferometer as a probe in situations where both general relativity and quantum mechanics are relevant.

Optical tweezer AIFs, like those discussed in Example 1, can be used to measure phases arising from proper time differences if the atom has at least two internal states. This is due to the fact that an atom with a single internal degree of freedom cannot produce a periodic signal necessary for measuring proper time. Interferometers employing only a single internal state can only measure effects stemming from a difference of the gravitational potential at different location, analogous to a gravitational Aharonov-Bohm effect. Such observations were made using atomic and neutron interferometry. Atomic clock interferometry was previously proposed as a method to measure proper time differences. However, the Inventor found that these proposals suffer from the same limitation of free falling AIF discussed in Example 1. This Example presents a guided ACIF, using optical tweezers at a magic wavelength, including an analysis of its available obeservables. The scheme inherits the advantages of the tweezer AIF, namely long probing duration and high position accuracy. Additionally, the measurement of the proper time difference is insensitive to relative intensity fluctuations, which makes it applicable to current tweezer technology.

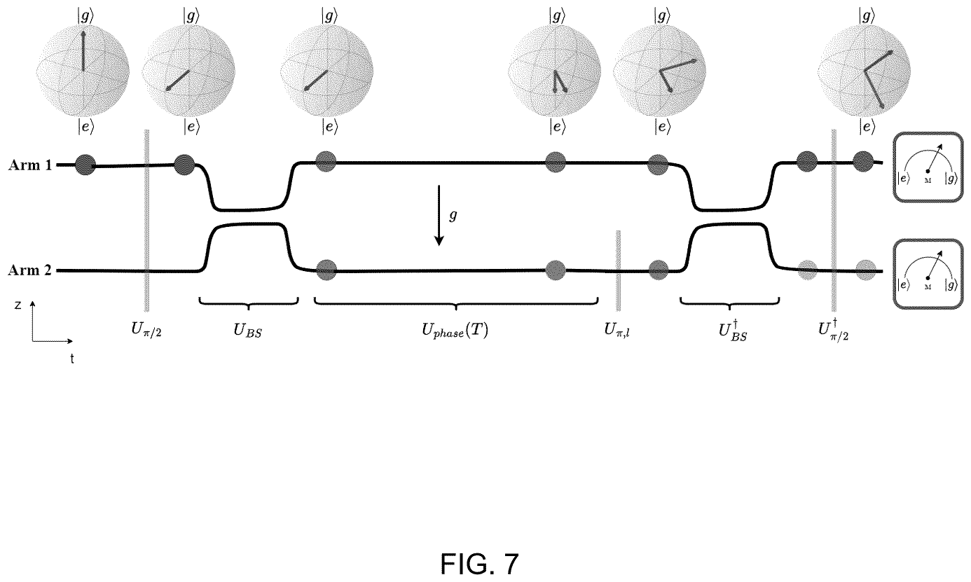

The guided ACIF scheme is depicted in FIG. 7. It is a modification of the scheme discussed in Example 1. It employs an atom which can be used as an atomic clock, and which has two long-lived internal states, denoted by |g [symbol] and |e [symbol] . The tweezers are selected to operate at the magic wavelength of the clock, where both clock states experience the same trap potential. The atom is initially prepared in the ground state |g [symbol] . Before the wave packet splitting stage, a π/2-pulse of an optical field, which is resonant with the |g [symbol] →|e [symbol] transition, is applied. This pulse generates the superposition (|g [symbol] +|e [symbol] )/√2.

Following the π/2-pulse, the spatial splitting of the clock wavefunction occurs using the two tweezers. As detailed in Example 1, achieving balanced splitting relies on adiabatic following to reach the symmetric ground state of a symmetric double-well potential. However, in the modified scheme the splitting occurs in the direction defined by gravity. The presence of a gravitational potential disrupts this spatial symmetry, potentially leading to imbalanced splitting. Numerical simulations indicate that Earth's gravitational potential results in a probability distribution of 44% and 56% for atoms being in the higher and lower arms, respectively, thus diminishing the visibility of interference fringes. To counteract this, applying a magnetic field gradient to negate the gravitational force during splitting and recombination is effective. Altering the depth of the tweezers during the operation can also compensate for the potential imbalance. Alternatively, conducting the splitting and recombining processes in a plane perpendicular to gravity and subsequently repositioning the arms at different heights can eliminate gravity's influence. Below, it is assumed that the imbalance in splitting is not significant, and the description is provided for the case of balanced atomic beam-splitters.

Each interferometer arm is moved to a different position in the gravitational field. Therefore, each part of the clock wavefunction experiences a different proper time. Before the recombining phase, a π-pulse on the |g [symbol] -|e [symbol] transition is applied to only one of the arms. The sequence ends with the spatial recombining of the wavefunction, as described in Example 1, followed by another π/2-pulse on the clock degrees of freedom to close the Ramsey sequence. The measured observables are the internal state of the atom and the spatial output port. As demonstrated below, both quantities are required for a measurement of proper time differences in a coherent superposition of an atom.

To proceed with the calculation, a unitary time-evolution operator approach is used. The vectors spanning the relevant Hilbert space are labeled by |k:l [symbol] , where k=g,e and l=1,2 denote the internal (clock) and external (paths) degrees of freedom, respectively. The evolution operators employed in the description of the interferometer are defined with respect to the following vectors:

$$\vert g ; 1 〉 ≡ ( 1 , 0 , 0 , 0 ) \\ \vert g ; 2 〉 ≡ ( 0 , 1 , 0 , 0 ) \\ \vert e ; 1 〉 ≡ ( 0 , 0 , 1 , 0 ) \\ \vert e ; 2 〉 ≡ ( 0 , 0 , 0 , 1 ) .$$

After the splitting and before the recombining, the energy difference ΔE (and the corresponding angular frequency ω₀) between the two clock states in path 2 are defined as

$$Δ E = {ℏω}_{0} = {E}_{e ; 2} - {E}_{g ; 2} .$$

It is assumed that there is a gravitational potential difference between the paths, Δφ, which gives rise to a difference in the proper time. This means that clocks following the different paths tick at a different rate. Therefore, the transition energy between the clock states must depend on the path, since it can be used as a clock. This is written explicitly as

$${E}_{e ; 1} - {E}_{g ; 1} = Δ E + ℏϵ$$

with ε=ω₀Δφ/c² being the angular frequency difference due to the gravitational redshift. The ACIF sequence allows determination of &. Note that the above expressions for assume that the energy difference between the clock states, E_{e; 2}-E_{g; 2} and for E_{e; 1}-E_{g; 1}, do not depend on the depth of the confining optical potential. This can be ensured by operating the trap at the magic wavelength.

To calculate the output of interferometric sequence, the unitary operations from which it is composed is defined. U_{BS} is the unitary time-evolution operator corresponding to the spatial wavefunction beam-splitter,

$${U}_{BS} = \frac{1}{\sqrt{2}} ( \begin{matrix}1 & 1 & 0 & 0 \\ 1 & - 1 & 0 & 0 \\ 0 & 0 & 1 & 1 \\ 0 & 0 & 1 & - 1\end{matrix} ) .$$

U_{π/2} corresponds to a π/2-pulse over the internal degree of freedom for both interferometer arms,

$${U}_{π / 2} = \frac{1}{\sqrt{2}} ( \begin{matrix}1 & 0 & - 1 & 0 \\ 0 & 1 & 0 & - 1 \\ 1 & 0 & 1 & 0 \\ 0 & 1 & 0 & 1\end{matrix} ) .$$

U_{π,l} represents a π-pulse over the internal degree of freedom in the lower arm only, the upper arm remains unchanged,

$${U}_{π , l} = \frac{1}{\sqrt{2}} ( \begin{matrix}1 & 0 & 0 & 0 \\ 0 & 0 & 0 & - 1 \\ 0 & 0 & 1 & 0 \\ 0 & 1 & 0 & 0\end{matrix} ) .$$

Uₚₕₐₛₑ(T) corresponds to the phase accumulation stage with a duration T,

$${U}_{phase} ( T ) = ( \begin{matrix}1 & 0 & 0 & 0 \\ 0 & {e}^{- i δ T} & 0 & 0 \\ 0 & 0 & {e}^{i ( Δ + ϵ ) T} & 0 \\ 0 & 0 & 0 & {e}^{i ( Δ - δ ) T}\end{matrix} ) ,$$

where a global phase was we omitted. The detuning between the two lower state is defined as δ=(E_{g; 2}-E_{g; 1})/ℏ, and the drive detuning is defined as Δ=ω−ω₀, with ω being the frequency of the field driving the transitions involving the internal states. The Equation for Uₚₕₐₛₑ is written in a rotating frame defined by the transformation

$${U}_{rot} ( T ) = ( \begin{matrix}1 & 0 & 0 & 0 \\ 0 & 1 & 0 & 0 \\ 0 & 0 & {e}^{i ω T} & 0 \\ 0 & 0 & 0 & {e}^{i ω T}\end{matrix} ) .$$

The final state after the ACIF sequence can be written as

$$\vert {ψ}_{f} 〉 = {U}_{rot}^{†} ( T ) {U}_{π / 2}^{†} {U}_{BS}^{†} {U}_{π , l} {U}_{phase} ( T ) {U}_{BS} {U}_{π / 2} \vert g ; 1 〉 .$$

Note that an initial Uᵣₒₜ(0) was omitted since it is an identity.

In previous proposals of ACIF schemes, there is only one available observable for measurements of the interference pattern of the atomic phase due to proper time differences between the arms-either the spatial output port, or the internal clock state. A particular feature of the tweezer ACIF is that it has two spatial output ports and complete Ramsey sequence over the clock states, both exhibit coherent oscillations. The probability of the atoms to exit the interferometer from the upper port is given by

$${P}_{1} ( {ψ}_{f} ) = {P}_{g ; 1} + {P}_{e ; 1} = {\vert 〈 g ; 1 \vert {ψ}_{f} 〉 \vert}^{2} + {\vert 〈 e ; 1 \vert {ψ}_{f} 〉 \vert}^{2} == \frac{1}{2} [ 1 - sin [ T ( \frac{{E}_{g ; 2} - {E}_{g ; 1}}{ℏ} - \frac{ϵ}{2} ) ] sin [ T ( Δ - \frac{ϵ}{2} ) ] ] . \qquad ( EQ . 3 )$$

An interferometric measurement according to some embodiments of the present invention can verify that the atom was indeed in a coherent superposition during the sequence. The result of EQ. (3) allows this, since it exhibits coherent oscillations between the exit ports as a function of T. In contrast, if the wave packet collapses randomly to one of the paths and the state becomes completely mixed, the exit port probability is P_{1,mixed}(ψ_{f})=½. This means that measuring oscillations around the probability of ½ as a function of time verifies the coherence of the atomic wavefunction.

When measuring the spatial output port, the exit probability depends on the difference between the eigenenergies: E_{g; 1}-E_{g; 2}. This dependency makes the observable vulnerable to relative intensity fluctuations of the trap beams. This issue can be addressed in more than one way. there are two strategies. In one strategy the laser intensity is stabile throughout the interferometer's duration to ensure minimal noise in this measurement. In conventional atomic interferometry, this is the sole approach since one needs to determine the relative phase between the paths, which is done by measuring the phase of the exit port oscillations.

In the system of the present embodiments, however, one only needs to verify the coherence of the split wave packet, which allows for a simpler approach. Intensity differences between the tweezers (intentional or unintentional) can introduce essentially a random phase in the first sine term of EQ. (3). Given x=½−A sin(ϕ) with A∈[0, 0.5] and the phase ϕ having a uniform random distribution in [0, 2ϕ], the probability distribution of x∈[0.5−A,0.5+A] is

$$P ( x ) = \frac{1}{π \sqrt{{A}^{2} - {( 0.5 - x )}^{2}}} .$$

In the case of EQ. (3), A=sin [T(Δ−ε/2)]/2 can be set by the choice of T and Δ while ε can be neglected. T and Δ are well controlled in the experiment, and therefore A can be regarded as constant. Thus, if the wave packet is coherently split and maintained, the distribution of the estimator of P₁ aligns with P (x) across a sufficiently large dataset. Since P₁ changes from run to run, it is preferred to estimate it with sufficient accuracy in each run. This can be done by executing interferometeric runs with several atoms. As show below, 10 atoms per run are sufficient to generate a clear distinction between coherent and non-coherent wave packet.

With this approach the gravitational redshift, ε, can be measured, because the probability of the atoms to be in the clock ground state at the end of the sequence (regardless of the output port) can be used as an additional observable. This is given by

$${P}_{g} ( {ψ}_{f} ) = {P}_{g ; 1} + {P}_{g ; 2} = {\vert 〈 g ; 1 \vert {ψ}_{f} 〉 \vert}^{2} + {\vert 〈 g ; 2 \vert {ψ}_{f} 〉 \vert}^{2} = \frac{1}{2} [ 1 + sin [ T ( Δ - \frac{ϵ}{2} ) ] sin [ T \frac{ϵ}{2} ] ] . \qquad ( EQ . 4 )$$

As demonstrated by EQ. 4, in contrast to the case of measuring the spatial output port, the probability to finish in the ground state depends only on Δ and ε, but not on the eigenenergies separately. This observable is therefore insensitive to relative fluctuations in the depths of the tweezers, which would shift the eigenenergies, but do not affect ω₀ and Δ, as long as the system operates at the magic wavelength. This means that for measuring the internal clock state, the main noise source limiting conventional guided AIFs is highly suppressed.

The probability to be in the ground state is the same whether the state is coherent or mixed. Therefore, it is preferred to measure both the spatial output port and the internal state of the atom. The measurement of the output port ensures the coherence of the atomic state, while the measurement of the internal state, which is robust in the presence of laser intensity noise, can be used to extract ε.

The redshift can be extracted from EQ. (4). By scanning the waiting time, T, the probability to find the atoms in the ground state oscillates due to interference of the two paths. The oscillation frequency of this interference pattern is set by the detuning of the driving field relative to the clock frequency, Δ, shifted by ε/2. For example, by choosing Δ>>2π/T, ε ensures that the oscillations can be observed by scanning the time from T to T+2π/Δ, during which the amplitude of the oscillations is substantially constant and given by

$$𝒱 = sin ( T \frac{ϵ}{2} ) .$$

Since this amplitude is determined by ε, it gives a straightforward way to extract its value. For small redshifts (Tε<<1), the visibility scales favorably linearly with ε:

$$𝒱 \approx T ϵ / 2.$$

This can be compared with a similar interferometric sequence, only without the π pulse in the lower path. In that case, the visibility of the interference pattern scales as

$$𝒱 \approx 1 - {( T ϵ )}^{2} / 8.$$

The linear scaling of the visibility in this scheme is advantageous, as further detailed below.

The measurable effect in a realistic experimental scenario is firstly estimated. The redshift is

$$ϵ = \frac{{ω}_{0}}{{c}^{2}} gh ,$$

with h being the height difference between the two interferometer arms. The atom are taken to be ¹⁷¹Yb, with the clock transition being ¹S₀-³P₀. The energy difference between the clock states corresponds to optical emission with a wavelength of approximately λ=578 nm. The magic wavelength of the tweezer trap for this atom is around λ_{magic}=759 nm.

The resilience of the measurement to differential intensity fluctuations depends on the stability of the tweezer wavelength. Deviations from the magic wavelength result in intensity-dependent light-shifts in the clock states frequencies, which in turn may reduce the accuracy. The tweezers's wavelength can be stabilized to the desired value with sub-MHz resolution, resulting in frequency noise in the order of 10⁻⁵ Hz. This noise level has no effect on the measurement accuracy, as we have confirmed through numerical simulations.

To reduce or minimize motional transitions in the tweezer during the optical pulse, the system optionally and preferably operates within the Lamb-Dicke regime. This regime is characterized by a small η=2πx₀/λ, where x₀ represents the spatial extent of the atomic wavefunction trapped in the tweezer. For a tweezer with a depth of 300 μK and a waist of σ=1 μm, the resulting Lamb-Dicke parameters are approximately η≈0.3 and 0.73 in the radial and axial directions, respectively. These values fall within the operational range for optical atomic clocks, demonstrating they are adequate for the interferometer. During the phase accumulation stage, in the absence of optical pulses, a deep trap is unnecessary. To lower the chance of spontaneous emission from the tweezer light during this extended phase, the depth of the tweezers is optionally and preferably reduced and then increased again just before the recombination stage.

It is assumed that a separation between the two tweezer arms is h=10 mm, aligned in the same direction as Earth's gravitational acceleration, g. Taking a phase accumulation duration of T=10 s, a visibility of about 0.02 is obtained. The oscillating signal appears on top of a background signal of 0.5. To estimate the number of runs required to clearly observe the gravitational effect, the Inventor performed a Monte Carlo simulation of the entire experiment. In the simulation, the detuning was set to Δ=2π·1000 Hz and N₁ different durations were scanned in the range T∈[10, 10+\2π/Δ]. For each of these durations, the Inventor simulated the measurement with Nₐ atoms according to EQs. (3) and (4) above. The procedure was then repeated N₂ times for each duration to get an estimator of P_{g}(T) and P₁(T). The fringe of the former was fitted, and from the extracted visibility, the value of ε was found. Additionally, included intensity fluctuations between the tweezer arms were included by adding a random energy shift on the order of ℏΔ between the E_{g; 2} and E_{g; 1} states.

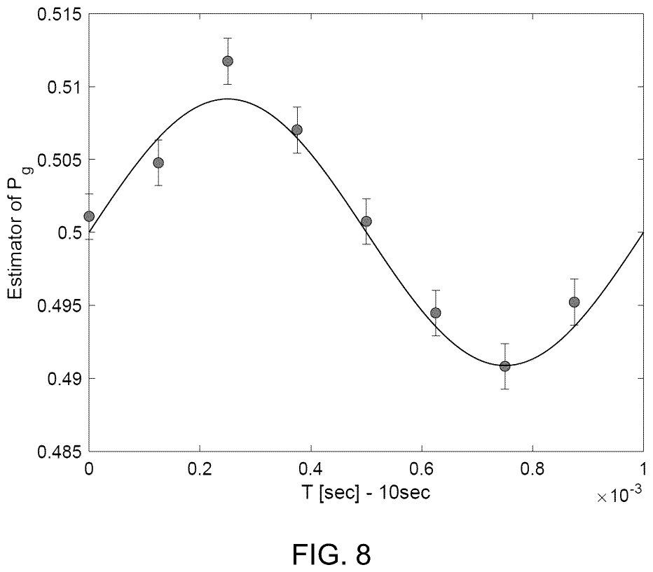

A typical result of the simulation is shown in FIG. 8. In this example, Nₐ was set to be 20 atoms per run and N₂ was set to be 5000 repetitions. Factoring in an overhead of 5 seconds for each run, the total duration per run extends to 15 seconds. Scanning the fringe at N₁=8 different phases, as shown in FIG. 8, requires about a week of data collection. With these parameters, the fringe contrast was clearly visible, and the relative accuracy (one standard deviation) in determining ε was approximately 9%. The accuracy of the extracted ε was not compromised by the intensity fluctuations between the tweezers. Table 3, below, presents the relative accuracy for other realistic choices of parameters. In all cases, the accuracy allows unambiguous determination of a gravitational redshift effect.

TABLE 3

| Number of atoms | Repetitions per phase | Probing duration | | Relative accuracy |
|---|---|---|---|---|
| in a run (Nₐ) | and duration (N₂) | (T) [sec] | total runtime | in extracting ε |
| 100 | 5000 | 1 | ~2.8 days | 38.1% |
| 100 | 10000 | 1 | ~5.6 days | 28.4% |
| 10 | 1000 | 10 | ~1.4 days | 28.4% |
| 10 | 5000 | 10 | ~7 days | 12.4% |
| 20 | 5000 | 10 | ~7 days | 8.8% |
| 100 | 1000 | 10 | ~1.4 days | 9% |
| 100 | 5000 | 10 | ~7 days | 3.8% |
| 100 | 10000 | 10 | ~13.9 days | 2.7% |

In all cases in Table 3, a separation of h=10 mm between the interferometer arms in Earth's gravitational field was assumed. Similar to FIG. 8, the number of different phases was fixed to N₁=8. The total runtime takes into account an overhead of 5 seconds per run. The relative accuracy is defined as one standard deviation of the extracted values of ε over an ensemble of 1000 Monte Carlo simulation runs, normalized by the theoretical value of ε.

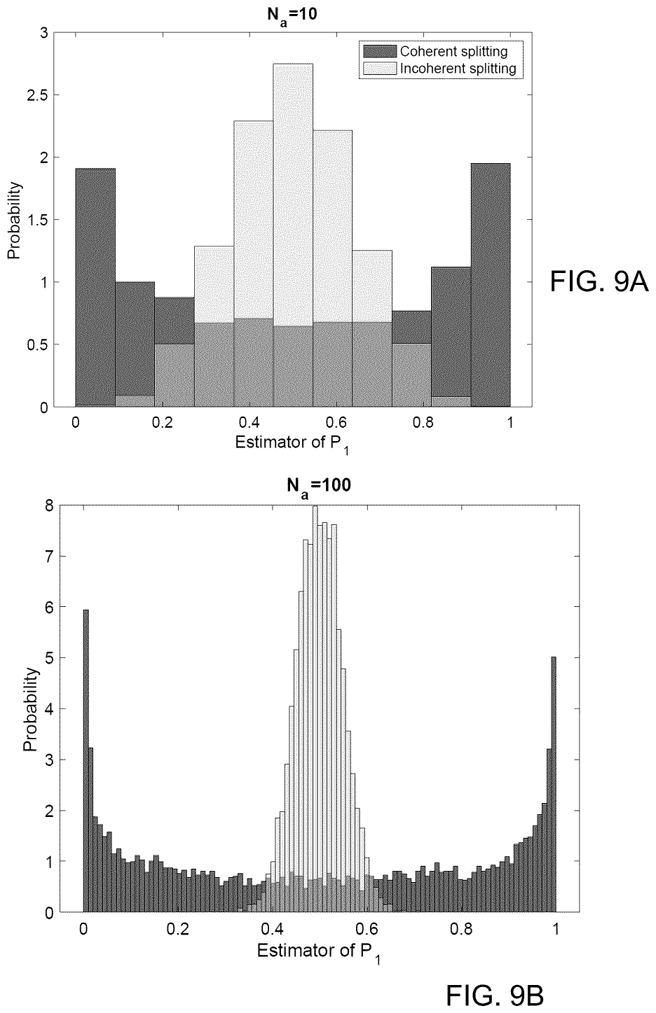

The Monte Carlo simulations allow to test the technique of the present embodiments for verifying the coherent splitting. The random fluctuations introduced in the relative ground state energies translate into essentially a random phase of the first sine term in EQ. (4). FIGS. 9A-B show the histogram of the estimator of P₁ (darker shading) for 10 (FIG. 9A) and 100 (FIG. 9B) atoms in each run. For comparison, the histogram obtained if a collapse randomly occurs to one of the two arms is also shown (brighter shading). The coherent and incoherent histograms are markedly different. In particular, the probability of finding highly unbalanced splitting between the output ports is negligibly small when the wave packet decoheres, while it is maximal if it maintains its coherence. These results prove that the coherence of the wave packet can be verified even in the presence of strong intensity fluctuations and with a relatively small number of atoms.

Operating two optical Ramsey-like pulses coherently over a span of 10 seconds implies a use of a narrow linewidth of the clock laser. This is similar to the case of light-pulse interferometers using the optical transition in ⁸⁸Sr. Indeed, known laser systems built for atomic clocks can reach a linewidth below 10 mHz, achieving coherence times that exceed 10 seconds. As demonstrated such a laser is sufficient for the estimation of the redshift measurement according to some embodiments of the present invention.

By using of commercially available laser systems, clock coherence times on the order of 1-5 seconds can be achieved. As shown in Table 3, by using 100 atoms in each run 28.4% relative accuracy can be achieved in less than 6 days of integration.

### Discussion

This Example introduced a guided atomic clock interferometer approach, using the tweezer interferometer of the present embodiments, in case in which the atoms have with two internal states. This technique employs optical laser pulses to create a superposition of the internal states and utilizes precise manipulation of the tweezers' position and intensity for the spatial adiabatic splitting and recombining of the wave packet. After completion of the interferometric scheme, the population within each clock state and exit port was record. The statistical distribution of the splitting between the two exit ports confirms the wave packet's spatial coherence throughout the experiment. Oscillations between the internal states reveal time dilation across the paths. In particular, these oscillations allow determining the gravitational redshift between the interferometer arms.

In the context of a clock interferometer, the gravitational field causes entanglement between the atom's internal state and its spatial wavefunction. In a larger scale scenario, a body composed of many such internal degrees of freedom can be viewed as multiple clocks operating at varying rates, influenced by the gravitational field. This variance in ticking rates leads to dephasing among the clocks and consequently reduction in coherence for the spatial wavefunction. This offers a potential explanation for decoherence in the classical limit that does not rely on interaction with environment. Using an ACIF according to some embodiments of the present invention allows testing the effect of gravity on the coherence of macroscopic objects.

The ACIF of the present embodiments can also be used for investigations of quantum mechanical theories, such as, but not limited to, quantum time. It is recognized that while in general relativity, the time is dynamic and dependent on the metric, the quantum mechanical theory treats time as a global parameter. Thus, the ACIF of the present embodiments can be used to determine whether the proper time is represented by a quantum operator, by examining deviations from the expected result of the ACIF experiment.

### Example 3

When an atom is very close to a surface, the interaction between the atom and the surface can be described as an attraction between the fluctuating atomic dipole and its mirror image. The Casimir-Polder (CP) potential in this case, which is also referred to as Lennard-Jones or van der Waals potential, scales as U_{CP}∝1/z³, where z is the distance to the surface. In the opposite limit, called the retarded limit, the potential scales as U_{CP}∝1/z⁴. The transition between these two regimes occurs at a typical length scale of 1˜100 nm. The exact CP force depends on the surface electrical properties, roughness, and temperature. Their precise measurement is important to test approximation methods in QED and as a means to understand material properties. Casimir forces are generally small, but they have a significant impact at the nanoscale, making them useful for nano-technology applications, specifically micro-electro-mechanical systems (MEMS).

The atomic interferometer of the present embodiments can be used to measure the phase shift induced by the CP interaction over a long duration, optionally and preferably providing a precise and model-independent measurement over a wide range of distances.

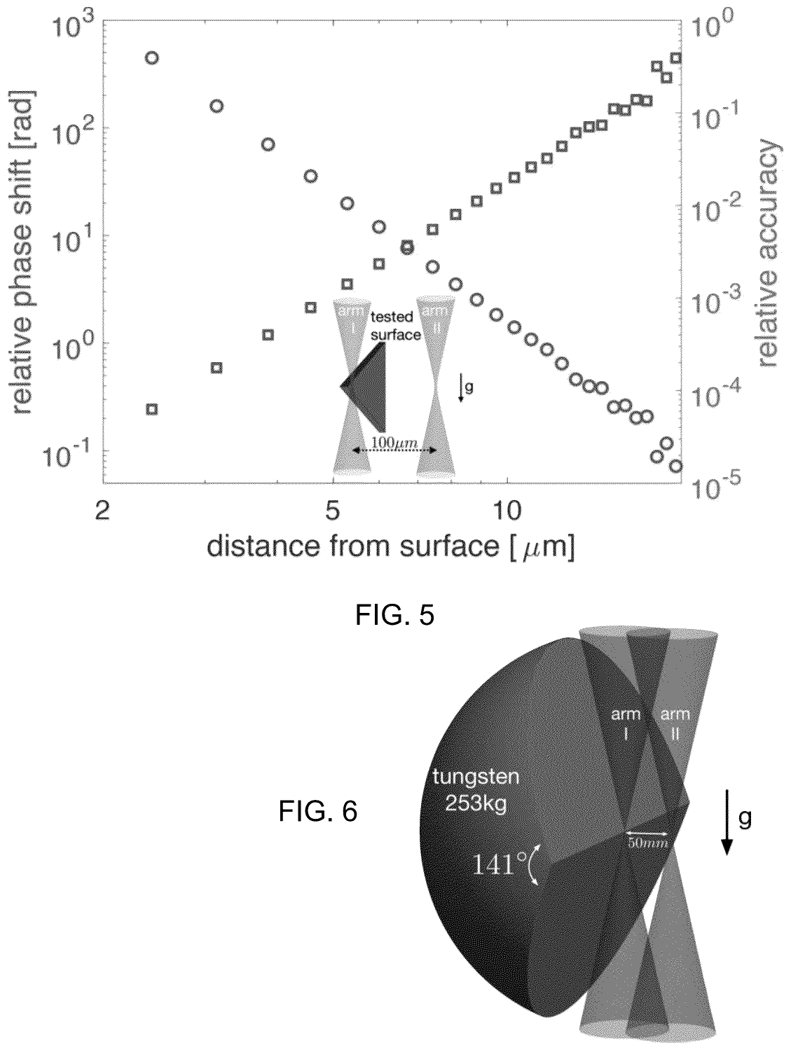

In these embodiments, one tweezer of the interferometer is positioned close to a surface, where it acquires a phase shift due to the Casimir-Polder potential. The second tweezer can be positioned at a position where the CP potential is negligible (e.g., about 100 micrometers away). The second tweezer thus acts as a reference. FIG. 5 shows a calculation of the relative phase shift acquired due to the CP potential versus the distance of the first tweezer from the surface. The phase shift is calculated in the retarded limit U_{CP}(z)=−C₄/z⁴, since the tweezer position z>>1, where 1≈118 nm. The constant C₄ was set to 1.64·10⁻⁵⁵ J m⁴. The calculation in FIG. 5 assumed operating parameters similar to the first row of Table 1 but with x=1 mm (T=10 s, 20 different phases, each repeated 10 times, for a total of 50 minutes of runtime). A phase uncertainty of about 41 mrad was added randomly to the calculated phase shift.

The calculation shows that with the tweezer interferometer optionally and preferably, the CP potential can be mapped in the region 1<<2<20 μm with very high precision. For example, at a distance of 5 μm, a measurement with relative precision of 0.0015 is estimated. It was verified that the non-additive effect of the tweezer on CP potential for the tweezer parameters is negligible.

It is advantageous to reduce the interference between the sample and the tweezer's Gaussian beam. To avoid clipping the tweezer beam, the sample can be shaped as a triangle (see inset of FIG. 5), allowing for the tweezer to be moved closer to the base as it is taken farther from the surface. This way, the ratio between the distance and the surface area is maintained, as well as the clear solid angle.

The typical size of the atomic wave-packet is about 100 nm and combined with the tweezers positioning accuracy, the position uncertainty is estimated at about 200 nm. The measurement can be done with any surface, whether it be metallic or dielectric. The mass of the samples can be made very small thanks to their two dimensional nature, ensuring that their gravitational potential is negligible. Disentangling between gravitational and surface forces is useful for investigation of non-Newtonian gravitational theories.

### Example 4

The tweezer atomic interferometer of the present embodiments has the ability to position a point-like test mass with sub-micrometer accuracy and to maintain that position for a prolonged duration. This Example provides a brief summary of the current understanding of the gravitational constant, focusing on the contributions made by measurements using the Kasevich-Chu interferometer. This Example also describes a measurement of G using the tweezer atomic interferometer of the present embodiments.

The value of Newton's gravitational constant, denoted by G, determines the strength of the gravitational force between two masses, and knowing its precise value is useful for a wide range of applications, including the study of celestial bodies and the prediction of the orbits of satellites and planets. There have been many attempts to measure G over the years, using a variety of techniques, including torsion balances, Cavendish balances, and spacecraft tracking. Still, G is the least known of all fundamental constants, with a slow improvement in its accuracy.

The 2018 Committee on Data of the International Science Council (CODATA) recommended a value of G=6.67430 (15)·10⁻¹¹ m³ kg⁻¹ s⁻², with a relative uncertainty of 2.2·10⁻⁵. This uncertainty is relatively large compared to that of other constants, such as the fine structure constant (1.5·10⁻¹⁰), the electron mass (3·10⁻¹⁰), and the vacuum electric permittivity (1.5·10⁻¹⁰). The reason is the relative weakness of gravity compared to the other forces.

The tweezer atomic interferometer of the present embodiments can be used for measuring G. The approach is illustrated in FIG. 6, benefits from the ability to position the test mass (atoms, in the present example) at a specific location for a long time. This Example uses a tungsten source, about 253 kg in mass, shaped as an angled cut from a sphere with a radius of 20 cm. This shape was designed to avoid clipping the tweezers' Gaussian beams. The atomic wave packets in the two arms of the interferometer can be positioned in a plane perpendicular to gravity that contains the center of the cut sphere. This cancels out the effect of Earth's gravitational potential in the interferometric measurement without a need for a dual interferometer setup. The wave packets can be placed at distances of r₁=1 mm and r₂=50 mm away from the center of the cut sphere. The distance r₂ is optionally and preferably as large as possible, and the value selected in this Example balances practical considerations with sensitivity.

The method can be executed alternately with and without the source mass to eliminate systematic deviations. The ability to move the tweezers at will facilitates that, as the mass can be left stationary while the tweezers are simply moved to a different location. The interferometer sequence can begin by splitting the atomic wave packet and moving the two tweezers to r₁ and r₂. The wave packets can be held in these positions for T=10 s before moving back and recombined. The fringe phase can be scanned by changing the duration T+ΔT, with |ΔT|<20 ms. For these exemplified experimental values, the phases accumulated by 40K atoms due to the sphere's gravitational potential are about 798 rad and about 615 rad for r₁ and r₂, respectively. With a single atom in each experiment, sampling 20 different phases across the fringe and repeating each one a plurality of times (e.g., 288 times which provide a total of one day of integration), the phase accuracy determination can be estimated at about 8·10⁻³ rad. If all other experimental parameters have small sufficient uncertainty, G can be measured with a relative accuracy of about 4.6·10⁻⁵. Running the same experiment with 100 atoms at a time (either in a single tweezer or in several in parallel) can improve the accuracy by another factor of 10. The method can employ ¹⁷¹Yb, which increases the relative phase shift and improve the relative accuracy by a factor of 4.275.

### APPENDIX

### Two-Tweezers Atomic Splitter

Following is a mathematical explanation of the operational principle of the two-tweezers atomic splitter according to some embodiments of the present invention.

The combined tweezers' potential V(x,d,Δ) is written as a sum of a symmetric function Vₛ(x,d)=Vₛ(−x,d) and an anti-symmetric function Vₐ(x,d)=−Vₐ (−x,d) multiplied by a detuning parameter 1≥Δ≥0, so that,

$$V ( x , d , Δ ) = {V}_{s} ( x , d ) + Δ \cdot {V}_{a} ( x , d ) ,$$

where the detuning parameter is varied from Δ=1 at the beginning of the process to Δ=0 at the end of the process.

Consider an unperturbed Hamiltonian of the form,

$${H}_{0} ( d ) = \frac{{ℏ}^{2}}{2 m} {∇}^{2} + {V}_{s} ( d ) + {V}_{a} ( d ) ,$$

and add to it, the perturbation potential (Δ−1)Vₐ(d), so that the full Hamiltonian becomes,

$$H ( d , Δ ) = {H}_{0} ( d ) + ( Δ - 1 ) {V}_{a} ( d ) .$$

The perturbation will now be used, denoting the eigenstates of H₀(d) with ϕ₁⁽⁰⁾ (x,d), ϕ₂ ⁽⁰⁾ (x,d), ϕ₃ ⁽⁰⁾ (x,d), . . . and the corresponding eigenvalues are E₁ ⁽⁰⁾ (d), E₂ ⁽⁰⁾ (d), E₃ ⁽⁰⁾ (d), . . . .

For simplicity, consider the two lowest eigenstates and describe the system with a two-level Hamiltonian, H(d,Δ)=H₀(d)+(Δ−1)Vₐ(d), where,

$${H}_{0} ( d ) = {E}_{1} ( d ) \vert {ϕ}_{1}^{( 0 )} ( d ) 〉 〈 {ϕ}_{1}^{( 0 )} ( d ) \vert + {E}_{2} ( d ) \vert {ϕ}_{2}^{( 0 )} ( d ) 〉 〈 {ϕ}_{2}^{( 0 )} ( d ) \vert$$

$$and$$

$${V}_{a} ( d ) = {\sum }_{j = 1 , 2} {V}_{jj} ( d ) \vert {ϕ}_{j}^{( 0 )} 〉 〈 {ϕ}_{j}^{( 0 )} \vert + {V}_{12}^{( 0 )} ( d ) \vert {ϕ}_{2}^{( 0 )} 〉 〈 {ϕ}_{1}^{( 0 )} \vert + h . c . ,$$

$$with ,$$

$${V}_{ij} ( d ) = 〈 {ϕ}_{j}^{( 0 )} ( d ) \vert {V}_{a} ( d ) \vert {ϕ}_{i}^{( 0 )} 〉 .$$

Using perturbation theory, the energy eigenvalues of H(d,Δ) are computed according to,

$${E}_{k} ( d , Δ ) = {E}_{k}^{( 0 )} ( d ) + ( Δ - 1 ) {V}_{kk} ( d ) ++ ( Δ - 1 ) {\sum }_{j \neq k} \frac{{\vert {V}_{jk} ( d ) \vert}^{2}}{{E}_{k}^{( 0 )} ( d ) - {E}_{j}^{( 0 )} ( d )} + …$$

with the resp. eigenstates, given by,

$${ϕ}_{k} ( d , Δ ) = {ϕ}_{k}^{( 0 )} ( d ) ++ ( Δ - 1 ) {\sum }_{j \neq k} \frac{{V}_{jk} ( d )}{{E}_{j}^{( 0 )} ( d ) - {E}_{k}^{( 0 )} ( d )} {ϕ}_{j}^{( 0 )} ( d ) + …$$

At t=0 the detuning parameter Δ(0) is set to 1 and the distance d(0) between the tweezers is large so that the coupling between the first and the second energy levels is practically zero, V₁₂(t=0)=0. The anti-symmetric potential Vₐ(x,d) is set to detune the potentials of the two tweezers to avoid degeneracy of the two lowest eigenstates. The detuning is selected such that for large d the first energy eigenstate is localized in the first tweezer, while the second energy eigenstate is localized in the second tweezer. This can be achieved by ensuring that the detuning potential Vₐ(x,d) does not exceed the energy difference between the energy levels of a single tweezer.

Then, as distance between the two tweezers d(t) is decreased, the transition amplitude V₁₂ (t) between the two energy levels of the system increases and becomes significant. At the same time the detunning parameter Δ(t) is lowered from one to zero. At time t_{f} when the detunning parameter is zero, the total potential of the tweezers V(x,d)=Vₛ(x,d) becomes a symmetric function. At this time the lowest energy eigenstate of the system is, |ϕ₁(x,t_{f}) [symbol] is symmetric and the second energy eigenstate |ϕ₂(x,t_{f}) [symbol] is anti-symmetric.

The adiabatic theorem guaranties that if d(t) and Δ(t) are changed in a sufficiently slow manner, the state of the system follows the eigenstates |ϕₓ (d(t),Δ(t) [symbol] in an adiabatic manner. Initially, at t=0, the lowest energy state of the system describes an atom located at the left tweezer. As the tweezers approach each other, the transition amplitude V₁₂(t) becomes large while at the same time Δ(t) is gradually lowered to zero. Avoided crossing effect (that occurs due to the fact that this two-level system has a finite non-zero transition amplitude |V₁₂(t)|>0), guarantees that the energy levels remain distinct and do not cross. Adiabaticity guaranties that the initial state |ϕ₁(d(0),Δ(0)) [symbol] of an atom in the left tweezer, is transformed adiabatically to a symmetric state |ϕ₁(d(t_{f}),Δ(t_{f})=0) [symbol] .

Also, if the atom is initially, e.g., in the right tweezer, its initial state is in the second energy level eigenstate |ϕ₂(d(0),Δ(0)) [symbol] and this state transforms adiabatically to the second anti-symmetric energy state |ϕ₂(d(t_{f}),Δ(t_{f})=0) [symbol] . Either way, this realizes an interferometer splitter. In the first case (an atom in the left tweezer) the phase between the two spacetime paths of atom is 0 and in the second case (an atom in the right Δ(0)>0 detuned tweezer) this phase is π.

Note that up to second order perturbation theory, the energy gap between the two lowest energy levels is given by,

$${E}_{2} ( d , Δ ) - {E}_{1} ( d , Δ ) == {E}_{2}^{( 0 )} ( d ) - {E}_{1}^{( 0 )} ( d ) ++ ( Δ - 1 ) ( {V}_{22} ( d ) - {V}_{11} ( d ) ) ++ 2 ( Δ - 1 ) \frac{{\vert {V}_{12} ( D , Δ ) \vert}^{2}}{{E}_{2}^{( 0 )} ( d ) - {E}_{1}^{( 0 )} ( d ) .}$$

As explained above, avoided crossing ensures that this energy gap E₂(d,Δ)−E₁(d,Δ) is always finite and positive.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

[1] B. P. Abbott, R. Abbott, T. D. Abbott, M. R. Abernathy, F. Acernese, K. Ackley, C. Adams, T. Adams, P. Addesso, R. X. Adhikari, et al. Observation of gravitational waves from a binary black hole merger. Physical Review Letters, 116 (6): 061102, 2016.
[2] Erika Andersson, Tommaso Calarco, Ron Folman, Mauritz Andersson, Bj\ddot{ } orn Hessmo, and J\ddot{ } org Schmiedmayer. Multimode interferometer for guided matter waves. Physical Review Letters, 88 (10): 100401, 2002.
[3] Peter Asenbaum, Chris Overstreet, Minjeong Kim, Joseph Curti, and Mark A. Kasevich. Atom-interferometric test of the equivalence principle at the 10-12 level. Phys. Rev. Lett., 125:191101, 2020.
[4] Dolev Bluvstein, Harry Levine, Giulia Semeghini, Tout T Wang, Sepehr Ebadi, Marcin Kalinowski, Alexander Keesling, Nishad Maskara, Hannes Pichler, Markus Greiner, et al. A quantum processor based on coherent transport of entangled atom arrays. Nature, 604 (7906): 451-456, 2022.
[5] Ch J Bord\acute{ } e. Atomic interferometry with internal state labelling. Physics letters A, 140 (1-2): 10-12, 1989.
[6] Antoine Browaeys and Thierry Lahaye. Many-body physics with individually controlled rydberg atoms. Nature Physics, 16 (2): 132-142, 2020.
[7] Daniel Carney, Holger M\ddot{ } uller, and Jacob M. Taylor. Using an atom interferometer to infer gravitational entanglement generation. PRX Quantum, 2 (3): 030330, 2021.
[8] V. P. Chebotayev, B. Ya. Dubetsky, A. P. Kasantsev, and V. P. Yakovlev. Interference of atoms in separated optical fields. J. Opt. Soc. Am. B, 2 (11): 1791-1798, 1985.
[9] Sheng-wey Chiow, Tim Kovachy, Hui-Chun Chien, and Mark A. Kasevich. 102⁻hk large area atom interferometers. Phys. Rev. Lett., 107:130403, 2011.
[10] Alexander D. Cronin, J\ddot{ } org Schmiedmayer, and David E. Pritchard. Optics and interferometry with atoms and molecules. Reviews of Modern Physics, 81 (3): 1051-1129, 2009.
[11] Savas Dimopoulos, Peter W. Graham, Jason M. Hogan, and Mark A. Kasevich. Testing general relativity with atom interferometry. Physical Review Letters, 98 (11): 111102, 2007.
[12] J. B. Fixler, G. T. Foster, J. M. McGuirk, and M. A. Kasevich. Atom interferometer measurement of the newtonian constant of gravity. Science, 315 (5808): 74-77, 2007.
[13] T M Graham, Y Song, J Scott, C Poole, L Phuttitarn, K Jooya, P Eichler, X Jiang, A Marra, B Grinkemeyer, et al. Multi-qubit entanglement and algorithms on a neutral-atom quantum computer. Nature, 604 (7906): 457-462, 2022.
[14] P. Hamilton, M. Jaffe, P. Haslinger, Q. Simmons, H. M\ddot{ } uller, and J. Khoury. Atominterferometry constraints on dark energy. Science, 349 (6250): 849-851, 2015.
[15] J. Schmiedmayer J.-F. Schaff, T. Langen. Interferometry with atoms. La Rivista del Nuovo Cimento, 37:509-589, 2014.
[16] Alec Jenkins, Joanna W. Lis, Aruku Senoo, William F. McGrew, and Adam M. Kaufman. Ytterbium nuclear-spin qubits in an optical tweezer array. Phys. Rev. X, 12:021027, 2022.
[17] Mark Kasevich and Steven Chu. Atomic interferometry using stimulated raman transitions. Phys. Rev. Lett., 67:181-184, 1991.
[18] A. M. Kaufman, B. J. Lester, C. M. Reynolds, M. L. Wall, M. Foss-Feig, K. R. A. Hazzard, A. M. Rey, and C. A. Regal. Two-particle quantum interference in tunnel-coupled optical tweezers. Science, 345 (6194): 306-309, 2014.
[19] Adam M. Kaufman and Kang-Kuen Ni. Quantum science with optical tweezer arrays of ultracold atoms and molecules. Nature Physics, 17 (12): 1324-1333, 2021.
[20] Jun Ke, Jie Luo, Cheng-Gang Shao, Yu-Jie Tan, Wen-Hai Tan, and Shan-Qing Yang. Combined test of the gravitational inverse-square law at the centimeter range. Physical Review Letters, 126 (21): 211101, 2021.
[21] D. W. Keith, M. L. Schattenburg, Henry I. Smith, and D. E. Pritchard. Diffraction of atoms by a transmission grating. Phys. Rev. Lett., 61:1580-1583, 1988.
[22] David W. Keith, Christopher R. Ekstrom, Quentin A. Turchette, and David E. Pritchard. An interferometer for atoms. Phys. Rev. Lett., 66:2693-2696, 1991.
[23] Shuo Ma, Alex P. Burgers, Genyue Liu, Jack Wilson, Bichen Zhang, and Jeff D. Thompson. Universal gate operations on nuclear spin qubits in an optical tweezer array of 171Yb atoms. Phys. Rev. X, 12:021028, 2022.
[24] A. A. Michelson and E. W. Morley. On the relative motion of the earth and the luminiferous ether. American Journal of Science, 34 (203): 333-345, 1887.
[25] Philip E. Moskowitz, Phillip L. Gould, Susan R. Atlas, and David E. Pritchard. Diffraction of an atomic beam by standing-wave radiation. Phys. Rev. Lett., 51:370-373, 1983.
[26] R. D. Newman, E. C. Berg, and P. E. Boynton. Tests of the gravitational inverse square law at short ranges. Space Science Reviews, 148 (1-4): 175-190, 2009.
[27] Matthew A. Norcia, Aaron W. Young, William J. Eckner, Eric Oelker, Jun Ye, and Adam M. Kaufman. Seconds-scale coherence on an optical clock transition in a tweezer array. Science, 366 (6461): 93-97, 2019.
[28] Cristian D Panda, Matt Tao, James Egelhoff, Miguel Ceja, Victoria Xu, and Holger M\ddot{ } uller. Quantum metrology by one-minute interrogation of a coherent atomic spatial superposition. arXiv: 2210.07289, 2022.
[29] Richard H. Parker, Chenghui Yu, Weicheng Zhong, Brian Estey, and Holger M\ddot{ } uller. Measurement of the fine-structure constant as a test of the standard model. Science, 360 (6385): 191-195, 2018.
[30] G. Rosi, F. Sorrentino, L. Cacciapuoti, M. Prevedelli, and G. M. Tino. Precision measurement of the newtonian gravitational constant using cold atoms. Nature, 510 (7506): 518-521, 2014.
[31] Friedhelm Serwane, Gerhard Z\ddot{ } urn, Thomas Lompe, T B Ottenstein, A N Wenz, and S Jochim. Deterministic preparation of a tunable few-fermion system. Science, 332 (6027): 336-338, 2011.
[32] Y. Shin, M. Saba, T. A. Pasquini, W. Ketterle, D. E. Pritchard, and A. E. Leanhardt. Atom interferometry with bose-einstein condensates in a double-well potential. Physical Review Letters, 92 (5): 050405, 2004.
[33] Benjamin M. Spar, Elmer Guardado-Sanchez, Sungjae Chi, Zoe Z. Yan, and Waseem S. Bakr. Realization of a fermi-hubbard optical tweezer array. Physical Review Letters, 128 (22): 223202, 2022.
[34] D. M. Stamper-Kurn, H.-J. Miesner, A. P. Chikkatur, S. Inouye, J. Stenger, and W. Ketterle. Reversible formation of a bose-einstein condensate. Phys. Rev. Lett., 81:2194-2197, 1998.
[35] Andreas Steffen, Andrea Alberti, Wolfgang Alt, Noomen Belmechri, Sebastian Hild, Micha l Karski, Artur Widera, and Dieter Meschede. Digital atom interferometer with single particle control on a discretized space-time geometry. Proceedings of the National Academy of Sciences, 109 (25): 9770-9774, 2012.
[36] Pippa Storey and Claude Cohen-Tannoudji. The feynman path integral approach to atomic interferometry. a tutorial. Journal de Physique II, 4 (11): 1999-2027, 1994.
[37] Giacomo Valtolina, Alessia Burchianti, Andrea Amico, Elettra Neri, Klejdja Xhani, Jorge Amin Seman, Andrea Trombettoni, Augusto Smerzi, Matteo Zaccanti, Massimo Inguscio, and Giacomo Roati. Josephson effect in fermionic superfluids across the BEC-BCS crossover. Science, 350 (6267): 1505-1508, 2015.
[38] N. V. Vitanov and B. W. Shore. Stimulated raman adiabatic passage in a two-state system. Physical Review A, 73 (5): 053402, 2006.
[39] J. A. C. Weideman and B. M. Herbst. Split-step methods for the solution of the nonlinear schrodinger equation. SIAM Journal on Numerical Analysis, 23 (3): 485-507, 1986.
[40] Victoria Xu, Matt Jaffe, Cristian D. Panda, Sofus L. Kristensen, Logan W. Clark, and Holger M\ddot{ } uller. Probing gravity by holding atoms for 20 seconds. Science, 366 (6466): 745-749, 2019.
[41] Zoe Z. Yan, Benjamin M. Spar, Max L. Prichard, Sungjae Chi, Hao-Tian Wei, Eduardo Ibarra-Garcia-Padilla, Kaden R. A. Hazzard, and Waseem S. Bakr. Two-dimensional programmable tweezer arrays of fermions. Phys. Rev. Lett., 129:123201, 2022.
[42] Shan-Qing Yang, Bi-Fu Zhan, Qing-Lan Wang, Cheng-Gang Shao, Liang-Cheng Tu, Wen-Hai Tan, and Jun Luo. Test of the gravitational inverse square law at millimeter ranges. Physical Review Letters, 108 (8): 081101, 2012.

What is claimed is:

1. An atomic interferometer system, comprising:
a plurality of optical tweezers each being configured to trap at least one atom therein;
an atom source system configured to release atoms;
a controller configured to control said optical tweezers to trap at least one atom released by said atom source system in one of said tweezers, to spatially split a wave function of said trapped atom between at least two of said tweezers, and to at least partially recombine said split atomic wave function in at least one of said tweezers; and
a measuring system, configured to measure wavefunction population in each of said tweezers and to display an output pertaining to said wavefunction populations.

2. The system according to claim 1, wherein each of said splitting and said recombination is adiabatic.

3. The system according to claim 1, wherein said controller is configured to gradually increase a tunneling rate among at least two of said tweezers and to gradually decrease detuning energy among said at least two of said tweezers, and then to gradually decrease said tunneling rate among while maintaining said detuning energy generally fixed, to thereby effect said splitting.

4. The system according to claim 3, wherein said controller is configured to increase said tunneling rate and to decrease said detuning energy simultaneously.

5. The system according to claim 1, wherein said controller is configured to effect said recombination a time period after said splitting, said time period being at least 10 seconds.

6. The system according to claim 1, wherein said controller is configured control said tweezers in pairs in a manner that said splitting and said recombination are executed among each pair independently from other pairs.

7. The system according to claim 1, wherein said atoms are fermionic atoms, wherein said atom source system is configured to release a plurality of atoms in a respective plurality of different discrete energy eigenstates, and wherein said controller is configured to control said optical tweezers to trap said plurality of atoms in one of said tweezers.

8. The system according to claim 7, wherein said atom source system comprises a laser cooling system configured to cool said atoms to form a Fermi gas, and said controller is configured to trap said Fermi gas in one of said tweezers and to reduce a number of atoms in said trapped Fermi gas by reducing a potential depth of said tweezer.

9. The system according to claim 1, wherein an atomic number of said atoms is at least 30.

10. The system according to claim 1, wherein said atoms are alkali group atoms.

11. The system according to claim 1, wherein said atoms are alkaline metal group atoms.

12. The system according to claim 1, wherein at least one of said tweezers has a waist of less than 10 μm in diameter.

13. A system for measuring a Casimir-Polder force between an atom and a surface, comprising the atomic interferometer system according to claim 1.

14. A system for mapping subsurface structures, comprising the atomic interferometer system according to claim 1.

15. The atomic interferometer system according to claim 1, in use for measuring at least one of time, acceleration, rotation, and gravity.

16. A system for detecting gravitational waves, comprising the atomic interferometer system according to claim 1.

17. A system for measuring atomic transitions, comprising the atomic interferometer system according to claim 1.

18. A system for seismic monitoring, comprising the atomic interferometer system according to claim 1.

19. An inertial navigation system, comprising the atomic interferometer system according to claim 1.

20. An atomic clock, comprising the atomic interferometer system according to claim 1.
